(19) United States
(12) Patent Application Publication
(10) Pub. No.: US 2004/0110809 A1
(43) Pub. Date: Jun. 10, 2004

(54) Metalloproteinase inhibitors

(76) Inventors: Matti Lepisto, Lund (SE); Magnus Munck Af Rosenschold, Lund (SE)

Correspondence Address: Janis K Fraser, Fish & Richardson, 225 Franklin Street, Boston, MA, 02110-2804, US

(21) Appl. No.: 10/471,499

(22) PCT Filed: Mar. 13, 2002
(86) PCT No.: PCT/SE02/00479

(30) Foreign Application Priority Data
Mar. 15, 2001 (SE) 0100903-4

(51) Int. Cl. A61K 31/426; A61K 31/421; A61K 31/4164

(52) U.S. Cl. 514/369; 514/376; 514/389; 548/183; 548/227; 548/317.1

(57) ABSTRACT

Compounds of the formula (I) useful as metalloproteinase inhibitors, especially as inhibitors of MMP12, wherein R5 is a bicyclic group.

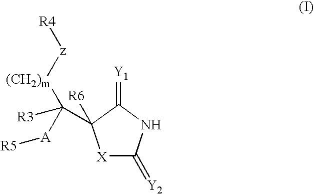

The present invention relates to compounds useful in the inhibition of metalloproteinases and in particular to pharmaceutical compositions comprising these, as well as their use.

The compounds of this invention are inhibitors of one or more metalloproteinase enzymes. Metalloproteinases are a superfamily of proteinases (enzymes) whose numbers in recent years have increased dramatically. Based on structural and functional considerations these enzymes have been classified into families and subfamilies as described in N. M. Hooper (1994) FEBS Letters 354:1-6. Examples of metalloproteinases include the matrix metalloproteinases (MMPs) such as the collagenases (MMP1, MMP8, MMP13), the gelatinases (MMP2, MMP9), the stromelysins (MMP3, MMP10, MMP11), matrilysin (MMP7), metalloelastase (MMP12), enamelysin (MMP19), the MT-MMPs (MMP14, MMP15, MMP16, MMP17); the reprolysin or adamalysin or MDC family which includes the secretases and sheddases such as TNF converting enzymes (ADAM10 and TACE); the astacin family which include enzymes such as procollagen processing proteinase (PCP); and other metalloproteinases such as aggrecanase, the endothelin converting enzyme family and the angiotensin converting enzyme family.

Metalloproteinases are believed to be important in a plethora of physiological disease processes that involve tissue remodelling such as embryonic development, bone formation and uterine remodelling during menstruation. This is based on the ability of the metalloproteinases to cleave a broad range of matrix substrates such as collagen, proteoglycan and fibronectin. Metalloproteinases are also believed to be important in the processing, or secretion, of biological important cell mediators, such as tumour necrosis factor (TNF); and the post translational proteolysis processing, or shedding, of biologically important membrane proteins, such as the low affinity IgE receptor CD23 (for a more complete list see N. M. Hooper et al., (1997) Biochem J. 321:265-279).

Metalloproteinases have been associated with many diseases or conditions. Inhibition of the activity of one or more metalloproteinases may well be of benefit in these diseases or conditions, for example: various inflammatory and allergic diseases such as, inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema, dermatitis); in tumour metastasis or invasion; in disease associated with uncontrolled degradation of the extracellular matrix such as osteoarthritis; in bone resorptive disease (such as osteoporosis and Paget's disease); in diseases associated with aberrant angiogenesis; the enhanced collagen remodelling associated with diabetes, periodontal disease (such as gingivitis), corneal ulceration, ulceration of the skin, post-operative conditions (such as colonic anastomosis) and dermal wound healing; demyelinating diseases of the central and peripheral nervous systems (such as multiple sclerosis); Alzheimer's disease; extracellular matrix remodelling observed in cardiovascular diseases such as restenosis and atheroscelerosis; asthma; rhinitis; and chronic obstructive pulmonary diseases (COPD).

MMP12, also known as macrophage elastase or metalloelastase, was initially cloned in the mouse by Shapiro et al (1992, Journal of Biological Chemistry 267: 4664) and in man by the same group in 1995. MMP-12 is preferentially expressed in activated macrophages, and has been shown to be secreted from alveolar macrophages from smokers (Shapiro et al, 1993, Journal of Biological Chemistry, 268: 23824) as well as in foam cells in atherosclerotic lesions (Matsumoto et al, 1998, Am J Pathol 153: 109). A mouse model of COPD is based on challenge of mice with cigarette smoke for six months, two cigarettes a day six days a week. Wildtype mice developed pulmonary emphysema after this treatment. When MMP12 knock-out mice were tested in this model they developed no significant emphysema, strongly indicating that MMP-12 is a key enzyme in the COPD pathogenesis. The role of MMPs such as MMP12 in COPD (emphysema and bronchitis) is discussed in Anderson and Shinagawa, 1999, Current Opinion in Anti-inflammatory and Immunomodulatory Investigational Drugs 1(1): 29-38. It was recently discovered.that smoking increases macrophage infiltration and macrophage-derived MMP-12 expression in human carotid artery plaques Kangavari (Matetzky S, Fishbein M C et at., Circulation 102:(18), 36-39 Suppl. S, Oct. 31, 2000).

MMP13, or collagenase 3, was initially cloned from a cDNA library derived from a breast tumour [J. M. P. Freije et al. (1994) Journal of Biological Chemistry 269(24):16766-16773]. PCR-RNA analysis of RNAs from a wide range of tissues indicated that MMP13 expression was limited to breast carcinomas as it was not found in breast fibroadenomas, normal or resting mammary gland, placenta, liver, ovary, uterus, prostate or parotid gland or in breast cancer cell lines (T47-D, MCF-7 and ZR75-1). Subsequent to this observation MMP13 has been detected in transformed epidermal keratinocytes [N. Johansson et al., (1997) Cell Growth Differ. 8(2):243-250], squamous cell carcinomas [N. Johansson et al., (1997) Am. J. Pathol. 151(2):499-508] and epidermal tumours [K. Airola et al., (1997) J. Invest. Dermatol. 109(2):225-231]. These results are suggestive that MMP13 is secreted by transformed epithelial cells and may be involved in the extracellular matrix degradation and cell-matrix interaction associated with metastasis especially as observed in invasive breast cancer lesions and in malignant epithelia growth in skin carcinogenesis.

Recent published data implies that MMP13 plays a role in the turnover of other connective tissues. For instance, consistent with MMP13's substrate specificity and preference for degrading type II collagen [P. G. Mitchell et al., (1996) J. Clin. Invest. 97(3):761-768; V. Knauper et al., (1996) The Biochemical Journal 271 :1544-1550], MMP13 has been hypothesised to serve a role during primary ossification and skeletal remodelling [M. Stahle-Backdahl et al., (1997) Lab. Invest. 76(5):717-728; N. Johansson et al., (1997) Dev. Dyn. 208(3):387-397], in destructive joint diseases such as rheumatoid and osteo-arthritis [D. Wernicke et al., (1996) J. Rheumatol. 23:590-595; P. G. Mitchell et al., (1996) J. Clin. Invest. 97(3):761-768; O. Lindy et al., (1997) Arthritis Rheum 40(8):1391-1399]; and during the aseptic loosening of hip replacements [S. Imai et al., (1998) J. Bone Joint Surg. Br. 80(4):701-710]. MMP13 has also been implicated in chronic adult periodontitis as it has been localised to the epithelium of chronically inflamed mucosa human gingival tissue [V. J. Uitto et al., (1998) Am. J. Pathol 152(6):1489-1499] and in remodelling of the collagenous matrix in chronic wounds [M. Vaalamo et al., (1997) J. Invest. Dermatol. 109(1):96-101].

MMP9 (Gelatinase B; 92 kDa TypeIV Collagenase; 92 kDa Gelatinase) is a secreted protein which was first purified, then cloned and sequenced, in 1989 [S. M. Wilhelm et al (1989) J. Biol Chem. 264 (29): 17213-17221; published erratum in J. Biol Chem. (1990) 265 (36): 22570]. A recent review of MMP9 provides an excellent source for detailed information and references on this protease: T. H. Vu & Z. Werb (1998) (In: Matrix Metalloproteinases. 1998. Edited by W. C. Parks & R. P. Mecham. pp115-148. Academic Press. ISBN 0-12-545090-7). The following points are drawn from that review by T. H. Vu & Z. Werb (1998).

The expression of MMP9 is restricted normally to a few cell types, including trophoblasts, osteoclasts, neutrophils and macrophages. However, it's expression can be induced in these same cells and in other cell types by several mediators, including exposure of the cells to growth factors or cytokines. These are the same mediators often implicated in initiating an inflammatory response. As with other secreted MMPs, MMP9 is released as an inactive Pro-enzyme which is subsequently cleaved to form the enzymatically active enzyme. The proteases required for this activation in vivo are not known. The balance of active MMP9 versus inactive enzyme is further regulated in vivo by interaction with TIMP-1 (Tissue Inhibitor of Metalloproteinases-1), a naturally-occurring protein. TIMP-1 binds to the C-terminal region of MMP9, leading to inhibition of the catalytic domain of MMP9. The balance of induced expression of ProMMP9, cleavage of Pro- to active MMP9 and the presence of TIMP-1 combine to determine the amount of catalytically active MMP9 which is present at a local site. Proteolytically active MMP9 attacks substrates which include gelatin, elastin, and native Type IV and Type V collagens; it has no activity against native Type I collagen, proteoglycans or laminins.

There has been a growing body of data implicating roles for MMP9 in various physiological and pathological processes. Physiological roles include the invasion of embryonic trophoblasts through the uterine epithelium in the early stages of embryonic implantation; some role in the growth and development of bones; and migration of inflammatory cells from the vasculature into tissues.

MMP-9 release, measured using enzyme immunoassay, was significantly enhanced in fluids and in AM supernatants from untreated asthmatics compared with those from other populations [Am. J. Resp. Cell & Mol. Biol., (November 1997) 17(5):583-591]. Also, increased MMP9 expression has been observed in certain other pathological conditions, thereby implicating MMP9 in disease processes such as COPD, arthritis, tumour metastasis, Alzheimer's, Multiple Sclerosis, and plaque rupture in atherosclerosis leading to acute coronary conditions such as Myocardial Infarction.

MMP-8 (collagenase-2, neutrophil collagenase) is a 53 kD enzyme of the matrix metalloproteinase family that is preferentially expressed in neutrophils. Later studies indicate MMP-8 is expressed also in other cells, such as osteoarthritic chondrocytes [Shlopov et al, (1997) Arthritis Rheum, 40:2065]. MMPs produced by neutrophils can cause tissue remodelling, and hence blocking MMP-8 should have a positive effect in is fibrotic diseases of for instance the lung, and in degradative diseases like pulmonary emphysema. MMP-8 was also found to be up-regulated in osteoarthritis, indicating that blocking MMP-8 may also be beneficial in this disease.

MMP-3 (stromelysin-1) is a 53 kD enzyme of the matrix metalloproteinase enzyme family. MMP-3 activity has been demonstrated in fibroblasts isolated from inflamed gingiva [Uitto V. J. et al, (1981) J. Periodontal Res., 16:417-424], and enzyme levels have been correlated to the severity of gum disease [Overall C. M. et al, (1987) J. Periodontal Res., 22:81-88]. MMP-3 is also produced by basal keratinocytes in a variety of chronic ulcers [Saarialho-Kere U. K. et al, (1994) J. Clin. Invest., 94:79-88]. MMP-3 mRNA and protein were detected in basal keratinocytes adjacent to but distal from the wound edge in what probably represents the sites of proliferating epidermis. MMP-3 may thus prevent the epidermis from healing. Several investigators have demonstrated consistent elevation of MMP-3 in synovial fluids from rheumatoid and osteoarthritis patients as compared to controls [Walakovits L. A. et al, (1992) Arthritis Rheum., 35:35-42; Zafarullah M. et al, (1993) J. Rheumatol., 20:693-697]. These studies provided the basis for the belief that an inhibitor of MMP-3 will treat diseases involving disruption of extracellular matrix resulting in inflammation due to lymphocytic infiltration, or loss of structural integrity necessary for organ function.

A number of metalloproteinase inhibitors are known (see for example the review of MMP inhibitors by Beckett R. P. and Whittaker M., 1998, Exp. Opin. Ther. Patents, 8(3):259-282). Different classes of compounds may have different degrees of potency and selectivity for inhibiting various metalloproteinases.

Whittaker M. et al (1999, Chemical Reviews 99(9):2735-2776) review a wide range of known MMP inhibitor compounds. They state that an effective MMP inhibitor requires a zinc binding group or ZBG (functional group capable of chelating the active site zinc(II) ion), at least one functional group which provides a hydrogen bond interaction with. the enzyme backbone, and one or more side chains which undergo effective van der Waals interactions with the enzyme subsites. Zinc binding groups in known MMP inhibitors include carboxylic acid groups, hydroxamic acid groups, sulfhydryl or mercapto, etc. For example, Whittaker M. et al discuss the following MMP inhibitors:

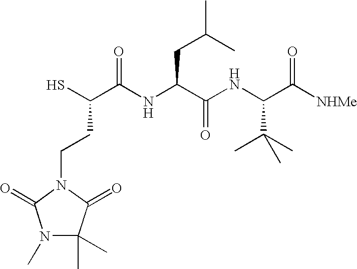

The above compound entered clinical development. It has a mercaptoacyl zinc binding group, a trimethylhydantoinylethyl group at the P1 position and a leucinyl-tert-butyllglycinyl backbone.

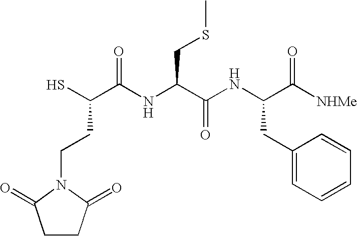

The above compound has a mercaptoacyl zinc binding group and an imide group at the P1 position.

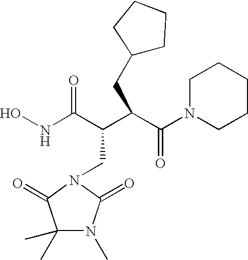

The above compound was developed for the treatment of arthritis. It has a non-peptidic succinyl hydroxamate zinc binding group and a trimethylhydantoinylethyl group at the P1 position.

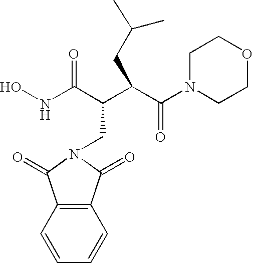

The above compound is a phthalimido derivative that inhibits collagenases. It has a non-peptidic succinyl hydroxamate zinc binding group and a cyclic imide group at P1. Whittaker M. et al also discuss other MMP inhibitors having a P1 cyclic imido group and various zinc binding groups (succinyl hydroxamate, carboxylic acid, thiol group, phosphorous-based group).

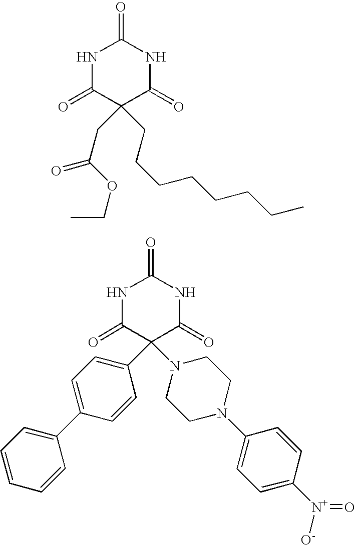

The above compounds appear to be good inhibitors of MMP8 and MMP9 (PCT patent applications WO9858925, WO9858915). They have a pyrimidin-2,3,4-trione zinc binding group.

The following compounds are not known as MMP inhibitors:

Japanese patent number 5097814 (1993) describes a method of preparing compounds useful as intermediates for production of antibiotics, including the compound having the formula:

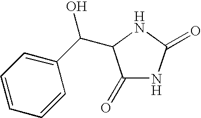

Morton et al (1993, J Agric Food Chem 41(1): 148-152) describe preparation of compounds with fungicidal activity, including the compound having the formula:

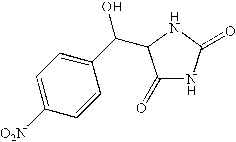

Dalgatov, D et al (1967, Khim. Geterotsikl. Soedin. 5:908-909) describe synthesis of the following compound without suggesting a use for the compound:

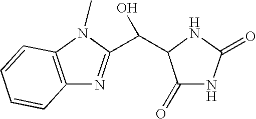

Crooks, P et al (1989, J. Heterocyclic Chem. 26(4):1113-17) describe synthesis of the following compounds that were tested for anticonvulsant activity in mice:

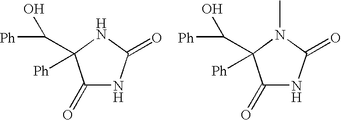

Gramain, J. C et al (1990) Recl. Trav. Chim. Pays-Bas 109:325-331) describe synthesis of the following compound:

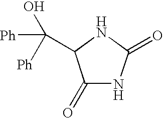

Japanese patent number 63079879 (1988) describes a method for the synthesis of intermediates en route to important amino acids. The following compounds have been used as starting materials:

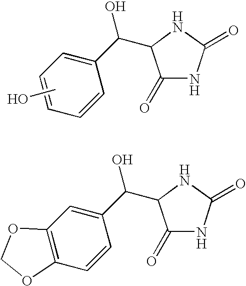

Wolfe, J et al (1971, Synthesis 6:310-311) describe synthesis of the following compound without suggesting a use for the compound:

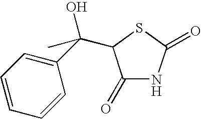

Moharram et al (1983, Egypt J. Chem. 26:301-11) describe the following compounds:

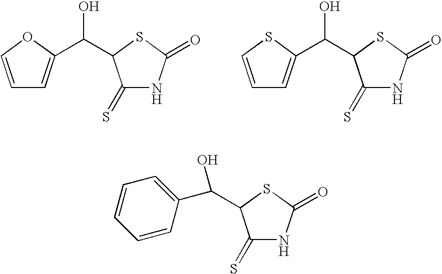

Hungarian patent number 26403 (1983) describes the synthesis and use as food additive of the following compound:

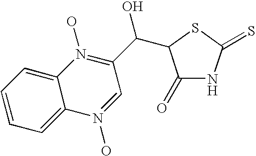

We have now discovered a new class of compounds that are inhibitors of metalloproteinases and are of particular interest in inhibiting MMPs such as MMP-12. The compounds are metalloproteinase inhibitors having a metal binding group that is not found in known metalloproteinase inhibitors. In particular, we have discovered compounds that are potent MMP12 inhibitors and have desirable activity profiles. The compounds of this invention have beneficial potency, selectivity and/or pharmacokinetic properties.

The metalloproteinase inhibitor compounds of the invention comprise a metal binding is group and one or more other functional groups or side chains characterised in that the metal binding group has the formula (k)

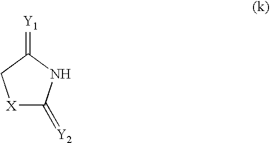

wherein X is selected from NR1, O, S;

Y1 and Y2 are independently selected from O, S;

R1 is selected from H, alkyl, haloalkyl;

Any alkyl groups outlined above may be straight chain or branched; any alkyl group outlined above is preferably (C1-7)alkyl and most preferably (C1-6)alkyl.

A metalloproteinase inhibitor compound is a compound that inhibits the activity of a metalloproteinase enzyme (for example, an MMP). By way of non-limiting example the inhibitor compound may show IC50s in vitro in the range of 0.1-10000 nanomolar, preferably in the range of 0.1-1000 nanomolar.

A metal binding group is a functional group capable of binding the metal ion within the active site of the enzyme. For example, the metal binding group will be a zinc binding group in MMP inhibitors, chelating the active site zinc(II) ion. The metal binding group of formula (k) is based on a five-membered ring structure and is preferably a hydantoin group, most preferably a -5 substituted 1-H,3-H-imidazolidine-2,4-dione.

In a first aspect of the invention we now provide compounds of the formula I

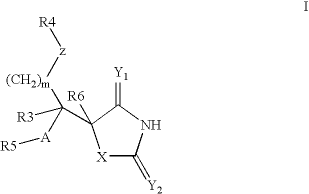

wherein

X is selected from NR1, O, S;

Y1 and Y2 are independently selected from O, S;

Z is selected from NR2, O, S;

m is 0 or 1;

A is selected from a direct bond, (C1-6)alkyl, (C1-6) alkenyl, (C1-6)haloalkyl, or (C1-6)heteroalkyl containing a hetero group selected from N, O, S, SO, SO2 or containing two hetero groups selected from N, O, S, SO, SO2 and separated by at least two carbon atoms;

R1 is selected from H, alkyl, haloalkyl;

R2 is selected from H, alkyl, haloalkyl;

R3 and R6 are independently selected from H, halogen (preferably F), alkyl, haloalkyl, alkoxyalkyl, heteroalkyl, cycloalkyl, aryl, alkyl-cycloalkyl, alkyl-heterocycloalkyl, heteroalkyl-cycloalkyl, heteroalkyl-heterocycloalkyl, cycloalkyl-alkyl, cycloalkyl-heteroalkyl, heterocycloalkyl-alkyl, heterocycloalkyl-heteroalkyl, alkylaryl, heteroalkyl-aryl, heteroaryl, alkylheteroaryl, heteroalkyl-heteroaryl, arylalkyl, aryl-heteroalkyl, heteroaryl-alkyl, heteroaryl-heteroalkyl, bisaryl, aryl-heteroaryl, heteroaryl-aryl, bisheteroaryl, cycloalkyl or heterocycloalkyl comprising 3 to 7 ring atoms, wherein the alkyl, heteroalkyl, aryl, heteroaryl, cycloalkyl or heterocycloalkyl radicals may be optionally substituted by one or more groups independently selected from hydroxy, alkyl, heteroalkyl, cycloalkyl, heterocycloalkyl, aryl, heteroaryl, halo, haloalkyl, hydroxyalkyl, alkoxy, alkoxyalkyl, haloalkoxy, haloalkoxyalkyl, carboxy, carboxyalkyl, alkylcarboxy, amino, N-alkylamino, N,N-dialkylamino, alkylamino, alkyl(N-alkyl)amino, alkyl(N,N-dialkyl)amino, amido, N-alkylamido, N,N-dialkylamido, alkylamido, alkyl(N-alkyl)amido, alkyl(N,N-dialkyl)amido, alkylcarbamate, alkylcarbamide, thiol, sulfone, sulfonamino, alkylsulfonamino, arylsulfonamino, sulfonamido, haloalkyl sulfone, alkylthio, arylthio, alkylsulfone, arylsulfone, aminosulfone, N-alkylaminosulfone, N,N-dialkylaminosulfone, alkylaminosulfone, arylaminosulfone, cyano, alkylcyano, guanidino, N-cyano-guanidino, thioguanidino, amidino, N-aminosulfon-amidino, nitro, alkylnitro, 2-nitro-ethene-1,1-diamine;

R4 is selected from H, alkyl, hydroxyalkyl, haloalkyl, alkoxyalkyl, haloalkoxy, aminoalkyl, amidoalkyl, thioalkyl;

R5 is a bicyclic or tricyclic group comprising two or three ring structures each of 3 to 7 ring atoms independently selected from cycloalkyl, aryl, heterocycloalkyl or heteroaryl, with each ring structure being independently optionally substituted by one or more substituents independently selected from halogen, thiolo, thioalkyl, hydroxy, alkylcarbonyl, haloalkoxy, amino, N-alkylamino, N,N-dialkylamino, cyano, nitro, alkyl, haloalkyl, alkoxy, alkyl sulfone, alkylsulfonamido, haloalkyl sulfone, alkylamido,alkylcarbamate, alkylcarbamide, carbonyl, carboxy, wherein any alkyl radical within any substituent may itself be optionally substituted by one or more groups independently selected from halogen, hydroxy, amino, N-alkylamino, N,N-dialkylamino, alkylsulfonamino, alkylcarboxyamino, cyano, nitro, thiol, alkylthiol, alkylsulfono, alkylaminosulfono, alkylcarboxylate, amido, N-alkylamido, N,N-dialkylamido, alkylcarbamate, alkylcarbamide, alkoxy, haloalkoxy, carbonyl, carboxy;

R5 is a bicyclic or tricyclic group wherein each ring structure is joined to the next ring structure by a direct bond, by —O—, by —S—, by —NH—, by (C1-6)alkyl, by (C1-6)haloalkyl, by (C1-6)heteroalkyl, by (C1-6)alkenyl, by (C1-6)alkynyl, by sulfone, by carboxy(C1-6)alkyl, or is fused to the next ring structure;

Optionally R2 and R4 may join to form a ring comprising up to 7 ring atoms or R3 and R6 may join to form a ring comprising up to 7 ring atoms;

Any heteroalkyl group outlined above or below is a hetero atom-substituted alkyl containing one or more hetero groups independently selected from N, O, S, SO, SO2, (a hetero group being a hetero atom or group of atoms);

Any heterocycloalkyl or heteroaryl group outlined above or below contains one or more hetero groups independently selected from N, O, S, SO, SO2;

Any alkyl, alkenyl or alkynyl groups outlined above or below may be straight chain or branched; unless otherwise stated, any alkyl group outlined above is preferably (C1-7)alkyl and most preferably (C1-6)alkyl;

Provided that:

when X is NR1, R1 is H, Y1 is O, Y2 is O, Z is O, m is 0, A is a direct bond, R3 is H, R4 is H and R6 is H, then R5 is not n-methylbenzimidazole, or 5-(benzo[1,3]dioxol-5-yl;

when X is S, at least one of Y1 and Y2 is O, m is 0, A is a direct bond, R3 is H or methyl, R6 is H or methyl, then R5 is not quinoxaline-1,4-dioxide.

Preferred compounds of the formula I are those wherein any one or more of the following apply:

X is NR1;

At least one of Y1 and Y2 is O; especially both Y1 and Y2 are O;

Z is O;

m is 0;

A is a direct bond;

R1 is H, (C1-3)allyl or (C1-3)haloalkyl; especially R1 is H or (C1-3)alkyl; most especially R1 is H;

R3 is H, alkyl or haloalkyl; especially R3 is H, (C1-6)alkyl or (C1-6)haloalkyl;

R4 is H, alkyl or haloalkyl; especially R4 is H, (C1-6)alkyl or (C1-6)haloalkyl; most especially R4 is H;

R5 is a bicyclic group comprising two optionally substituted ring structures each of 5 or 6 ring atoms and independently selected from cycloalkyl, aryl, heterocycloalkyl or heteroaryl; especially R5 comprises two aryl or heteroaryl 5 or 6 membered rings; more especially R5 is an optionally substituted biphenyl such as para-biphenyl, or para-phenoxyphenyl;

R6 is H, alkyl, hydroxyalkyl, aminoalkyl, cycloalkyl-alkyl, alkyl-cycloalkyl, arylalkyl, alkylaryl, heteroalkyl, heterocycloalkyl-alkyl, alkyl-heterocycloalkyl, heteroaryl-alkyl or heteroalkyl-aryl; especially R6 is alkyl, aminoalkyl or heteroaryl-alkyl.

Particular compounds of the invention include compounds of formula I wherein:

At least one of Y1 and Y2 is O (preferably both Y1 and Y2 are O), and X is NH, and m is 0; or

At least one of Y1 and Y2 is O, and X is NH, and Z is O, and A is a direct bond, and R3 and R4 are independently selected from H, alkyl or haloalkyl; or

Both Y1 and Y2 are O, and X is NH, and m is 0, and Z is O, and R4 is H.

In a further aspect of the invention we now provide compounds of the formula Ib

Formula Ib:

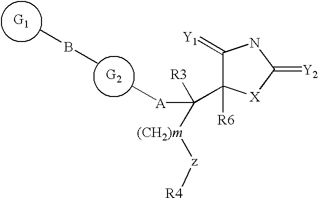

wherein

X is selected from NR1, O, S;

Y1 and Y2 are independently selected from O, S;

Z is selected from NR2, O, S;

m is 0 or 1;

A is selected from a direct bond, (C1-6)alkyl, (C1-6)haloalkyl, or (C1-6) heteroalkyl containing a hetero atom selected from O, S;

B is selected from a direct bond, —O—, —S—, —NH—, amide, carbamate, carbonyl, (C1-6)alkyl, (C1-6)haloalkyl, (C2-6)alkenyl, (C2-6)alkynyl, or (C1-6)heteroalkyl containing a hetero atom selected from O, S;

R1 is selected from H, (C1-3)allyl or (C1-3)haloalkyl;

R2 is selected from H, (C1-3)alkyl or (C1-3)haloalkyl;

R3 is selected from H, (C1-3)alkyl or (C1-3)haloalkyl;

R4 is selected from H, (C1-3)alkyl or (C1-3)haloalkyl;

R6 is selected from H, alkyl, heteroalkyl, (C3-7)cycloalkyl, (C3-7)heterocycloalkyl, (C3-7)aryl, (C3-7)heteroaryl, alkyl-(C3-7)cycloalkyl, alkyl-(C3-7)heterocycloalkyl, alkyl-(C3-7)aryl, alkyl-(C3-7)heteroaryl, heteroalkyl-(C3-7)cycloalkyl, heteroalkyl-(C3-7)heterocycloalkyl, heteroalkyl-(C3-7)aryl, heteroalkyl-(C3-7)heteroaryl, (C3-7)cycloalkyl-alkyl, (C3-7)heterocycloalkyl-alkyl, (C3-7)ary-alkyl, (C3-7)heteroaryl-alkyl, (C3-7)cycloalkyl-heteroalkyl, (C3-7)heterocycloalkyl-heteroalkyl, (C3-7)aryl-heteroalkyl, (C3-7)heteroaryl-heteroalkyl;

in R6 the alkyl, heteroalkyl, aryl, heteroaryl, cycloalkyl or heterocycloalkyl radicals may be optionally substituted by one or more groups independently selected from hydroxy, alkyl,halo, haloalkyl, hydroxyalkyl, alkoxy, alkoxyalkyl, haloalkoxy, haloalkoxyalkyl, carboxy, carboxyalkyl, alkylcarboxy, amino, N-alkylamino, N,N-dialkylamino, alkylamino, alkyl(N-alkyl)amino, alkyl(N,N-dialkyl)amino, amido, N-alkylamido, N,N-dialkylamido, alkylamido, alkyl(N-alkyl)amido, alkyl(N,N-dialkyl)amido, alkylcarbamate, alkylcarbamide, thiol, sulfone, sulfonamino, alkylsulfonamino, arylsulfonamino, sulfonamido, haloalkyl sulfone, alkylthio, arylthio, alkylsulfone, arylsulfone, aminosulfone, N-alkylaminosulfon, N,N-dialkylaminosulfone, alkylaminosulfone, arylaminosulfone, cyano, alkylcyano, guanidino, N-cyano-guanidino, thioguanidino, amidino, N-aminosulfon-amidino, nitro, alkylnitro, 2-nitro-ethene-1,1-diamine;

either G1 is a monocyclic group and G2 is selected from a monocyclic group and a bicyclic group, or G1 is a bicyclic group and G2 is a monocyclic group, wherein the monocyclic group comprises one ring structure and the bicyclic group comprises two ring structures either fused together or joined together by B as defined above, each ring structure having up to 7 ring atoms and being independently selected from cycloalkyl, aryl, heterocycloalkyl or heteroaryl, with each ring structure being independently optionally substituted by one or more substituents independently selected from halogen, thiolo, thioalkyl, hydroxy, alkylcarbonyl, haloalkoxy, amino, N-alkylamino, N,N-dialkylamino, cyano, nitro, alkyl, haloalkyl alkoxy, alkyl sulfone,alkylsulfonamido, haloalkyl sulfone, alkylamido,alkylcarbamate, alkylcarbamide, wherein any alkyl radical within any substituent may itself be optionally substituted by one or more groups independently selected from halogen, hydroxy, amino, N-alkylamino, N,N-dialkylamino, alkylsulfonamino, cyano, nitro, thiol, alkylthiol, alkylsulfono, alkylaminosulfono, alkylcarboxylate, amido, N-alkylamido, N,N-dialkylamido, alkylcarbamate, alkylcarbamide,alkoxy, haloalkoxy;

Optionally R3 and R6 may join to form a ring comprising up to 7 ring atoms.

Preferred compounds of the formula Ib are those wherein any one or more of the following apply:

X is NR1;

At least one of Y1 and Y2 is O; especially both Y1 and Y2 are O;

Z is O;

m is 0;

A is a direct bond, (C1-6)alkyl or (C1-6)heteroalkyl containing a hetero atom selected from O, S;

B is a direct bond, acetylene, CON (amide), (C1-C4)alkyloxy, —O—, —S—or —NH—;

R1 is H or methyl;

R3 is H, (C1-3)alkyl or (C1-3)haloalkyl;

R4 is H, (C1-3)alkyl or (C1-3)haloalkyl.

Particularly preferred compounds of the formula Ib are those wherein:

X is NR1 and R1 is H; and

Y1 and Y2 are each O; and

Z is O; and

m is 0; and

A is a direct bond; and

B is selected from a direct bond, acetylene, —O—, —NH—, —S—, or CH₂O;

R3 is H; and

R4 is H.

Thus we provide compounds of the formula Ic

Formula Ic:

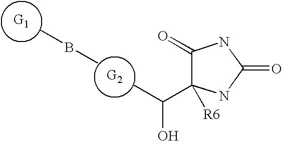

wherein

B is selected from a direct bond, acetylene, —O—, —NH—, —S—, or CH₂O;

each of G1, G2 and R6 is as defined for Formula Ib.

Preferred compounds of Formula Ic are those wherein any one or more of the following apply:

B is selected from a direct bond, —O—, —S—, or CH₂O; most preferably B is selected from a direct bond, —O—, CH₂O;

G2 is a monocyclic group comprising an aryl ring; most preferably G2 is phenyl;

G1 is a monocyclic or bicyclic group comprising at least one aryl ring; most preferably G1 is a monocyclic or bicyclic group comprising at least one five or six membered aryl ring;

R6 is selected from H, (C1-6)alkyl, (C1-6)heteroalkyl, heterocycloalkyl, heterocycloalkyl-(C1-6)alkyl, heteroaryl or heteroaryl-(C1-6)alkyl; preferred heteroaryls are pyridine, diazines (such as pyrimidine) or azoles (such as imidazol); preferred heterocycloalkyls are morpholino, piperidine or piperazine; preferred heteroalkyls are amino-(C1-C6)alkyl; preferred substituents on heteroaryls are halogen; preferred substituents on amines in heteroalkyls and heterocycloalkyls are alkyl, alkylsulfon, alkylaminocarbonyl or alkyloxycarbonyl.

Thus we provide compounds of the Formula Id

Formula Id:

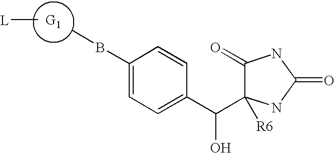

wherein

B is selected from a direct bond, O or CH₂O;

G1 is a monocyclic or bicyclic group comprising at least one five or six membered aryl ring;

R6 is H, alkyl, hydroxyalkyl, aminoalkyl, alkyl-carbamic acid alkyl ester, alkyl-alkyl-urea, alkylsulfonyl-alkyl, N-allyl-alkylsulfonamide, heteroaryl-alkyl;

L is selected from H, alkyl, haloalkyl, hydroxy, alkoxy, haloalkoxy, amino, alkylamino, amido, alkylamido, alkylcarbamate, alkylcarbamide, alkylsulfono, alkylsulfonamido,nitro, cyano, halo;

or L is a group:

T—U—V—

wherein V is attached to G1 and V is selected from CH₂, O, NCO, NCOO, NCON or NSO₂;

U is (C1-5)alkyl,

T is selected from hydroxy, alkoxy, cyano, amino, alkylamino, alkylsulfono, alkylsulfonamide, alkylcarbamate, alkylacarbamide, alkylamide, imidazolyl, triazolyl or pyrollidon.

Preferred compounds of Formula Id are those wherein any one or more of the following apply:

G1 is selected from phenyl, pyridyl, napthyl or quinoline;

R6 is selected from H, (C1-6)alkyl, hydroxy-(C1-6)alkyl, amino-(C1-6)alkyl, or heteoraryl-(C1-6)alkyl; most especially R6 is H, methyl, pyridinylmethyl, N-substituted amino-(C1-4)alkyl (preferred N-substituents are alkyl, alkylsulfonyl or carbamic acid alkyl ester);

L is selected from H, (C1-5)alkyl, (C1-5)haloalkyl, hydroxy, alkoxy, haloalkoxy, amino, (C1-5)alkylamino, amido, (C1-5)alkylamido, (C1-5)alkylcarbamate, (C1-5)alkylcarbamide, (C1-5)alkylsulfono, (C1-5)alkylsulfonamido, nitro, cyano, halo; or L is the group T—U—V— wherein V is as defined for the Formula Ic, U is unbranced (C1-5)alkyl, and T is selected from hydroxy, alkoxy, cyano, amino, (C1-3)alkylamino, (C1-3)alkylsulfono, (C1-3)alkylsulfonamide, (C1-3)alkylcarbamate, (C1-3)alkylacarbamide, (C1-3)alkylamide, imidazolyl, triazolyl or pyrollidon;

L is a meta or para substituent when G1 is a 6 membered ring.

Suitable values for R6 in compounds of formulae I, Ib, Ic, or Id include the following:

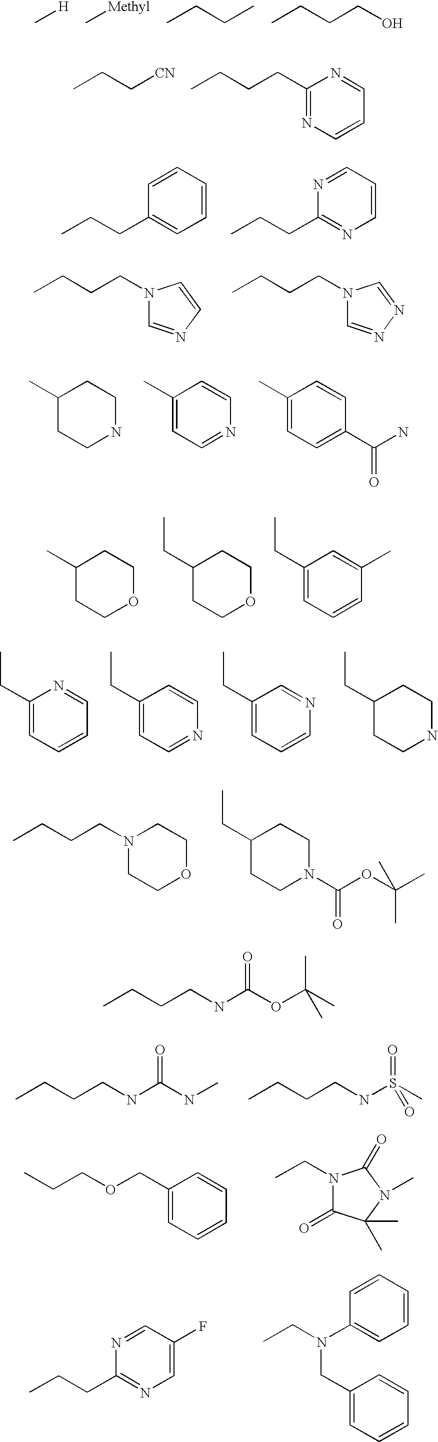

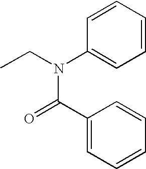

Suitable values for R5 in compounds of formula I or for G1B—G2 in compounds of formula Ib, Ic or Id include the following:

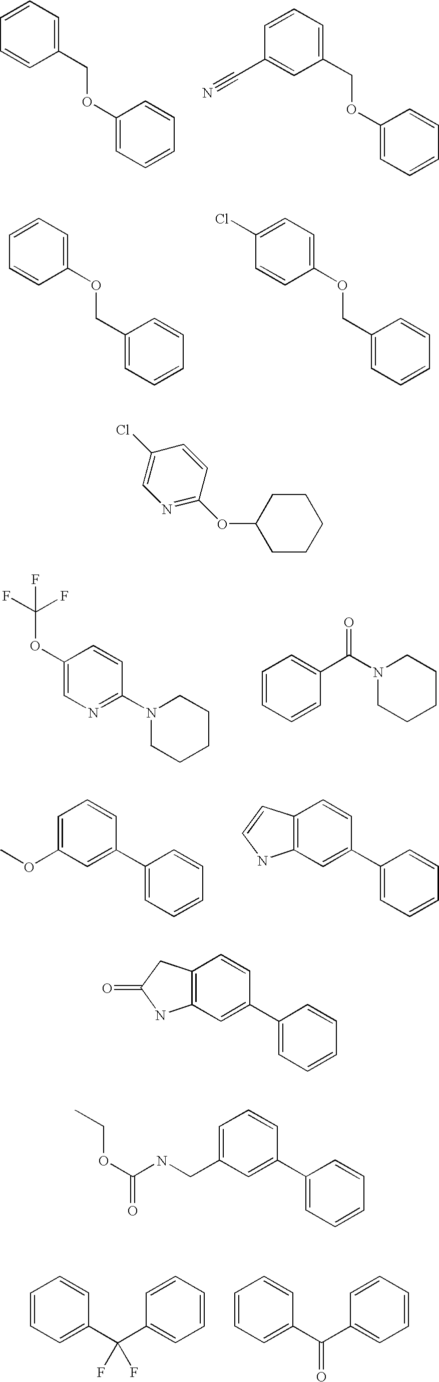

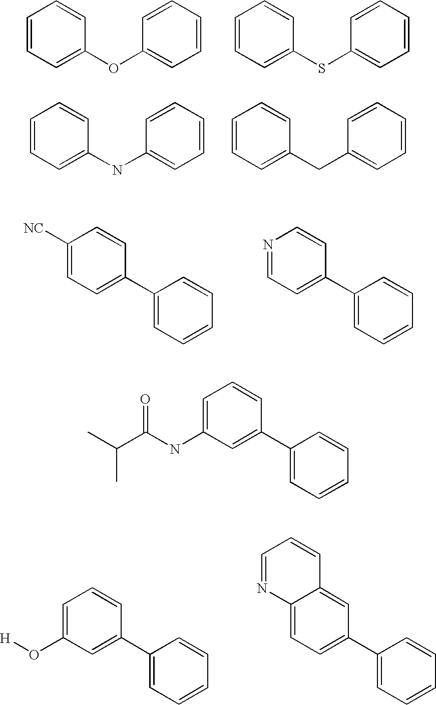

It will be appreciated that the particular substituents and number of substituents in compounds of the invention are selected so as to avoid sterically undesirable combinations.

Each exemplified compound represents a particular and independent aspect of the invention.

Where optically active centres exist in the compounds of the invention, we disclose all individual optically active forms and combinations of these as individual specific embodiments of the invention, as well as their corresponding racemates. Racemates may be separated into individual optically active forms using known procedures (cf. Advanced Organic Chemistry: 3rd Edition: author J March, p104-107) including for example the formation of diastereomeric derivatives having convenient optically active auxiliary species followed by separation and then cleavage of the auxiliary species.

It will be appreciated that the compounds according to the invention may contain one or more asymmetrically substituted carbon atoms. The presence of one or more of these asymmetric centres (chiral centres) in a compound of the invention can give rise to stereoisomers, and in each case the invention is to be understood to extend to all such stereoisomers, including enantiomers and diastereomers, and mixtures including racemic mixtures thereof.

Where tautomers exist in the compounds of the invention, we disclose all individual tautomers forms and combinations of these as individual specific embodiments of the invention.

As previously outlined the compounds of the invention are metalloproteinase inhibitors, in particular they are inhibitors of MMP12. Each of the above indications for the compounds of the invention represents an independent and particular embodiment of the invention.

Certain compounds of the invention are of particular use as inhibitors of MMP13 and/or MMP9 and/or MMP8 and/or MMP3. Certain compounds of the invention are of particular use as aggrecanase inhibitors ie. inhibitors of aggrecan degradation.

Compounds of the invention show a favourable selectivity profile. Whilst we do not wish to be bound by theoretical considerations, the compounds of the invention are believed to show selective inhibition for any one of the above indications relative to any MMP1 inhibitory activity, by way of non-limiting example they may show 100-1000 fold selectivity over any MMP1 inhibitory activity.

The compounds of the invention may be provided as pharmaceutically acceptable salts. These include acid addition salts such as hydrochloride, hydrobromide, citrate and maleate salts and salts formed with phosphoric and sulfuric acid. In another aspect suitable salts are base salts such as an alkali metal salt for example sodium or potassium, an alkaline earth metal salt for example calcium or magnesium, or organic amine salt for example triethylamine.

They may also be provided as in vivo hydrolysable esters. These are pharmaceutically acceptable esters that hydrolyse in the human body to produce the parent compound. Such esters can be identified by administering, for example intravenously to a test animal, the compound under test and subsequently examining the test animal's body fluids. Suitable in vivo hydrolysable esters for carboxy include methoxymethyl and for hydroxy include formyl and acetyl, especially acetyl.

In order to use a metalloproteinase inhibitor compound of the invention (including a compound of the formulae I, Ib, Ic, Id) or a pharmaceutically acceptable salt or in vivo hydrolysable ester thereof for the therapeutic treatment (including prophylactic treatment) of mammals including humans, it is normally formulated in accordance with standard pharmaceutical practice as a pharmaceutical composition.

Therefore in another aspect we provide a pharmaceutical composition which comprises a compound of the invention (such as a compound of the formulae I, Ib, Ic, Id) or a pharmaceutically acceptable salt or an in vivo hydrolysable ester thereof and pharmaceutically acceptable carrier.

The pharmaceutical compositions of this invention may be administered in standard manner for the disease or condition that it is desired to treat, for example by oral, topical, parenteral, buccal, nasal, vaginal or rectal adminstration or by inhalation. For these purposes the compounds of this invention may be formulated by means known in the art into the form of, for example, tablets, capsules, aqueous or oily solutions, suspensions, emulsions, creams, ointments, gels, nasal sprays, suppositories, finely divided powders or aerosols for inhalation, and for parenteral use (including intravenous, intramuscular or infusion) sterile aqueous or oily solutions or suspensions or sterile emulsions.

In addition to the compounds of the present invention the pharmaceutical composition of this invention may also contain, or be co-administered (simultaneously or sequentially) with, one or more pharmacological agents of value in treating one or more diseases or conditions referred to hereinabove.

The pharmaceutical compositions of this invention will normally be administered to humans so that, for example, a daily dose of 0.5 to 75 mg/kg body weight (and preferably of 0.5 to 30 mg/kg body weight) is received. This daily dose may be given in divided doses as necessary, the precise amount of the compound received and the route of administration depending on the weight, age and sex of the patient being treated and on the particular disease or condition being treated according to principles known in the art

Typically unit dosage forms will contain about 1 mg to 500 mg of a compound of this invention.

Therefore in a further aspect, we provide a compound of the formula I (especially a compound of the formulae Ib, Ic, Id) or a pharmaceutically acceptable salt or in vivo hydrolysable ester thereof for use in a method of therapeutic treatment of the human or animal body or for use as a therapeutic agent. We disclose use in the treatment of a disease or condition mediated by one or more metalloproteinase enzymes. In particular we disclose use in the treatment of a disease or condition mediated by MMP12 and/or MMP13 and/or MMP9 and/or MMP8 and/or MMP3 and/or aggrecanase; especially use in the treatment of a disease or condition mediated by MMP12 or MMP9; most especially use in the treatment of a disease or condition mediated by MMP12.

In yet a further aspect we provide a method of treating a metalloproteinase mediated disease or condition which comprises administering to a warm-blooded animal a therapeutically effective amount of a compound of the formulae I, Ib, Ic or Id or a pharmaceutically acceptable salt or in vivo hydrolysable ester thereof.

We also disclose the use of a compound of the formulae I, Ib, Ic, Id or a pharmaceutically acceptable salt or in vivo hydrolysable precursor thereof in the preparation of a medicament for use in the treatment of a disease or condition mediated by one or more metalloproteinase enzymes.

Metalloproteinase mediated diseases or conditions include asthma, rhinitis, chronic obstructive pulmonary diseases (COPD), arthritis (such as rheumatoid arthritis and osteoarthritis), atherosclerosis and restenosis, cancer, invasion and metastasis, diseases involving tissue destruction, loosening of hip joint replacements, periodontal disease, fibrotic disease, infarction and heart disease, liver and renal fibrosis, endometriosis, diseases related to the weakening of the extracellular matrix, heart failure, aortic. aneurysms, CNS related diseases such as Alzheimer's disease and Multiple Sclerosis (MS), hematological disorders.

Preparation of the Compounds of the Invention

In another aspect the present invention provides processes for preparing a compound of the formulae I, Ib, Ic, Id or a pharmaceutically acceptable salt or in vivo hydrolysable ester thereof, as described in (b) to (h) below (X, Y1, Y2, Z, m, A and R1-R6 are as hereinbefore defined for the compound of formula I).

(a) A compound of the invention may be converted to a salt, especially a pharmaceutically acceptable salt, or vice versa, by known methods; a salt, especially a pharmaceutically acceptable salt, of a compound of the invention may be converted into a different salt, especially a pharmaceutically acceptable salt, by known methods.

(b) Compounds of the invention in which Z═O and R4═H may be prepared by reacting a compound of the formula IIa with a compound of the formula IIIa or a suitably protected form of a compound of formula IIIa (as shown in Scheme 1), and optionally thereafter forming a pharmaceutically acceptable salt or in vivo hydrolysable ester thereof:

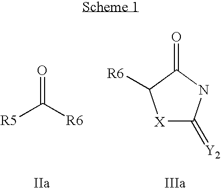

Aldehydes or ketones of formula IIa and compounds of formula IIIa in a suitable solvent are treated with a base, preferably in the temperature range from ambient temperature to reflux. Preferred base-solvent combinations include aliphatic amines such as trimethylamine, pyrrolidine or piperidine in solvents such as methanol, ethanol, tetrahydrofurane, acetonitrile or dimethylformamide, with addition of water when necessary to dissolve the reagents (Phillips, A P and Murphy, J G, 1951, J. Org. Chem. 16; or lithiumhexamethyldisilazan in tetrahydrofurane (Mio, S et al, 1991, Tetrahedron 47:2121-2132); or barium hydroxide octahydrate in isopropanol-water (Ajinomoto K K, 1993, Japanese Patent Number 05097814).

Preferably, when preparing compounds of the invention by this process, R3, R5 or R6 will not contain additional functionalities such as aldehydes, ketones, halogenated radicals or any other radicals well known to those skilled in the art which have the potential of interfering with, competing with or inhibiting the bond formation reaction.

It will be appreciated that many of the relevant staring materials are commercially or otherwise available or may be synthesised by known methods or may be found in the scientific literature.

To prepare compounds of the general formula IIIa (R6 as hereinbefore described), compounds of formula IIIa in which R6 is H may be reacted with an appropriate aldehyde or ketone followed by dehydration and subsequent reduction of the resulting double bond by methods which are well know to those skilled in the art.

(c) Compounds of the the invention in which Z═O, R4═H and X═N or NR1, especially specific stereoisomers thereof, may also be prepared as described for two of the four possible stereoisomers in Schemes 2 and 3 below.

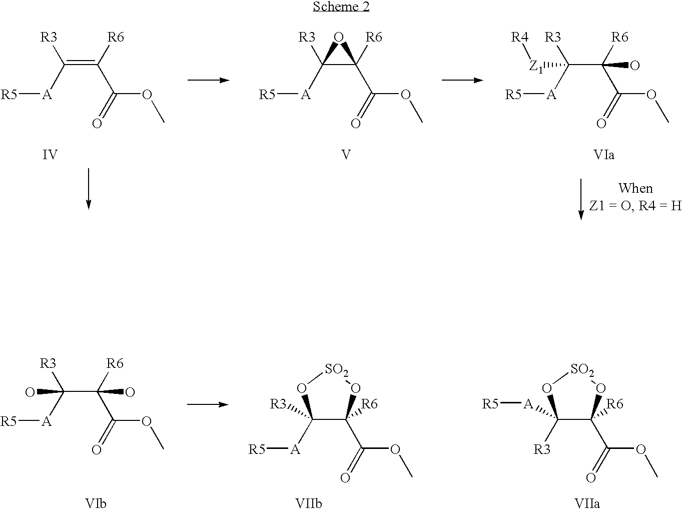

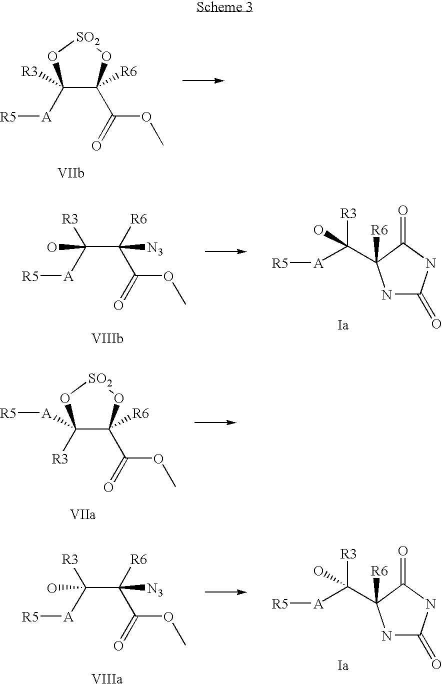

Starting from the propenoate derivatives of formula IV, via the diols VIa or VIb by either asymmetric epoxidation followed by regioselective opening with water, or asymmetric dihydroxylation. Depending on the chiral auxiliary in the epoxidation or dihydroxylation, either the shown stereoisomers or their enantiomers of the diols of formula VIa or VIb can be obtained. (For example, Ogino, Y. et al, 1991, Tetrahedron Lett. 2 (41):5761-5764; Jacobsen, E. N. et al, 1994, Tetrahedron, 50(15):4323-4334; Song, C. E. et al, 1997, Tetrahedron Asymmetry, 8 (6):841-844). Treatment with organic base and thionyl chloride and subsequent ruthenium tetroxide catalysed oxidation yields the cyclic sulfates VIIa and VIIb.

The cyclic sulfates of formula VIIa and VIIb are converted to the hydroxy azides (Scheme 3) of formula VIIIa and VIIIb by treatment with sodium azide in dimethylformamide followed by careful hydrolysis of the hemisulfate intermediates before aqueous work-up. (Gao, Sharpless, 1988, J.Am.Chem.Soc., 110:7538; Kim, Sharpless, 1989, Tetrahedron Lett., 30:655). The hydroxy azides of formula VIIIa and VIIIb are hydrolysed and reduced to the β-hydroxy-α-amino acids (not shown in Scheme 3), preferably hydrolysis with LiOH in THF followed by reduction with hydrogen sulfide, magnesium in methanol or organic phosphines by the Staudinger procedure. The β-hydroxy-α-amino acids in turn yield compounds of formula Ia upon treatment with cyanate and acid in aqueous media.

(d) Compounds of the invention in which Z═O and R4 is not H, especially specific stereoisomers thereof, may also be prepared as described for two of the four possible stereoisomers in Schemes 2 and 3. The compounds may be prepared by reacting the epoxides of formula V in Scheme 2 with an alcohol of formula R4-OH, yielding the alcohols Via. Subsequent conversion to the azides with phosphoazidate (Thompson, A. S. et al, 1993, J. Org. Chem. 58(22):5886-5888) yields the ether analogs of the azido esters VIIIa in Scheme 3, which can be carried through to the final products as described under process (c). The radical R4 in alcohols R4-OH and the radicals R3, R5 and R6 in may be suitably protected. The protecting groups can be removed as a last step after the conversion to the hydantoins of formula Ia.

(e) Compounds of the invention in which Z is S or NR2 and Y1 and/or Y2 is O, especially specific stereoisomers thereof, may also be prepared as described for two of the four possible stereoisomers in Schemes 2 and 3. The compounds may be synthesised by opening of the epoxides of formula V (Scheme2) with thiols R4-SH or amines R4-NH₂ and thereafter subjected to analogous transformations as described for the alcohols VIIIa and VIIIb in Scheme 3. When amines of R4-NH2 are used, it may be necessary to N-protect the intermediate amino alcohols, especially when the radical R4 is a n-alkyl group.

(f) Compounds of the invention in which X is S and Y1 and/or Y2 is O, especially specific stereoisomers thereof, may also be prepared as described for two of the four possible stereoisomers in Schemes 2 and 3. The compounds may be prepared by reacting the cyclic sulfates of formula VIIa or VIIb, or the α-hydroxy esters of formula VIa via their sulfonate esters, with thiourea and acid (1997, Japanese Patent number 09025273).

The propenoate derivatives of formula IV are widely accessible, eg from aldehydes and phosphonium or phosphonate derivatives of acetic acid via the Wittig or Homer-Emmons reaction (for example, van Heerden, P. S. et al, 1997, J. Chem. Soc., Perkin Trans. 1(8):141-1146).

(g) Compounds of the invention in which X═NR1 and R1═H may be prepared from reacting an appropriate substituted aldehyde or ketone of formula IId with ammonium carbonate and potassium cyanide in aqueous alcohols at 50-100° C. in a sealed vessel for 4-24 h.

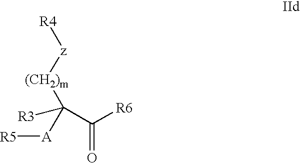

Preparations of some aldehydes or ketones of formula IId are described in:

Marte, A.-M. et al, Tetrahedron Lett., 1990, 31(18):2599-2602;

Kren, V. et al, 1993, J. Chem. Soc., Chem. Commun., 4:341-343;

Schmittel, M. et al, 1990, Angew. Chem., 102(10):1174-1176;

Chakraborty, R. et al, 1992, Synth. Commun., 2(11):1523;

Harder, T. et al,1994, Tetrahedron Lett, 35(40):7365-7368;

Ruder, S. M., 1992, Tetrahedron. Lett, 33(9):2621-2624;

Maeda, H. et al, 1997, Chem. Pharm. Bull., 45(11):1729-1733;

Montana, J. G. et al, 1994, J. Chem. Soc., Chem. Commun., 19:2289-2290;

Davis, B. R. et al, 1992, Aust. J. Chem. 45(5):865-875.

Some of the aldehydes or ketones are available through aldol reactions (m=1, Z═O):

Mahrwald, R, et al, 1998, J. Am. Chem. Soc., 120(2):413-414;

Auerbach, R. A., et al, 1988, Org. Synth., VI:692;

Mukaiyama, T.; 1977, Angew. Chem., (Int. Ed.) 16;

Shimizu, N. et al, 1983, Bull. Chem. Soc. Jpn., 56(12):853;

Maruoka, K. et al, 1986, J. Am. Chem. Soc., 108(13):3827.

Known preparation of compounds of formula IId are listed in Table 1 below:

TABLE 1

| Name (formyl 1^{st}, even when “non IUPAC”) | CAS number |
|---|---|
| 2-formyl-5-pyridin-3-yl furane | 38588-49-7 |
| 2-formyl-5-pyridin-2-y furane | 55484-36-1 |
| 5-formyl-2-phenyl oxazole | 92629-13-5 |
| 2-formyl-5-phenyl furane | 13803-39-9 |
| 2-formyl-3-methyl-5-phenyl furane | 160417-25-4 |
| 2-formyl-3-ethoxycarbonyl furane | 50800-39 |
| 2-formyl-5-phenyl-3,4-oxadiazole | 22816-01-9 |
| 2-formyl-5-phenyl oxazole | 96829-89-9 |
| 2-formyl-4-chloro-5-phenyl oxazole | 119344-57-9 |
| 2-formyl-4-chloro-2-pyridin-3-yl thiazole | 131969-58-9 |
| 2-formyl-5-pyridin-3-yl thiophene | 133531-43-8 |
| 2-formyl-5-pyridin-2-yl thiophene | 132706-12-8 |
| 2-formyl-5-pyridin-4-yl thiophene | 21346-36-1 |
| 5-formyl-2-phenyl thiazole | 1011-40-1 |
| 5-formyl-4-chloro-2-phenyl thiazole | 108263-77-0 |
| 5-formyl-4-methyl-2-phenyl thiazole | 55327-23-6 |
| 2-formyl-5-phenyl thiophene | 19163-21-4 |
| 2-formyl-3-methyl-5-phenyl thiophene | 1604417-30-1 |
| 4-formyl-2-pyridin-2-yl imidazole | 279251-08-0 |
| 2-formyl-1-methyl-5pyridin-3-yl pyrrole | 3614-77-5 |
| 4-formyl-2-pyridin-3-yl imidazole | 279251-09-1 |
| 4-formyl-2-pyridin-4-yl 1,3,4-triazole | 42786-73-2 |
| 4-formyl-2-pyridin-4-yl imidazole | 279251-10-4 |
| 4-formyl-5-methoxy-5-phenyl thiazole | 73725-36-7 |
| 4-formyl-5-ethoxycarbonyl-5-phenyl thiazole | 88469-73-2 |
| 4-formyl-5-ethoxycarbonyl-5-phenyl oxazole | 189271-85-0 |
| 2-formyl-3methyl-5-phenyl 1,3,4-triazole | 89060-36-6 |
| 4-formyl-1-methyl-2-phenyl imidazole | 94938-02-0 |
| 5-formyl-1-methyl-2-phenyl imidazole | 94938-03-1 |
| 4-formyl-1-butyl-2-phenyl imidazole | 198066-02-3 |
| 4-formyl-1-propyl-2-phenyl imidazole | 75378-63-1 |
| 5-formyl-1-butyl-2-phenyl imidazole | 198065-92-8 |
| 2-formyl-1-methyl-4-phenyl imidazole | 123511-51-3 |
| 4-formyl-2-phenyl-5-methyl oxazole | 70170-23-9 |
| 2-formyl-5-phenyl 1,3,4-triazole | 26899-64-9 |
| 4-formyl-2-phenyl-5-chloro imidazole | 60367-52-4 |
| 4-formyl-2-phenyl imidazole | 68282-47-3 |
| 4-formyl-2-phenyl-5-methyl imidazole | 68282-50-8 |
| 2-formyl-1-methyl-5-phenyl 1,3,4-triazole | 219600-03-0 |
| 2-formyl-4-phenyl imidazole | 56248-10-3 |
| 2-formyl-1-methyl-4-phenyl imidazole | 118469-06-0 |
| 2-formyl-5-phenyl pyrazole | 52179-74-5 |
| 2-formyl-3-methyl-5-phenyl pyrazole | 160417-28-7 |
| 2-formyl-3-ethoxycarbonyl-5-phenyl pyrazole | 63202-77-7 |
| 2-formyl-5-morfolin-1-yl furane | 3680-96-4 |
| 2-formyl-5-piperdin-1-yl furane | 22868-60-6 |
| 2-formyl-5-cyclohexyl furane | 14174-51-7 |
| 2-formyl-3-methyl-5-cyclohexyl furane | 160417-27-6 |

(h) Compounds of the invention may also be synthesized according to Scheme 4 below. Suitable target compounds include the substituted 5-(biphenyl4-yl-hydroxy-methyl)-imidazolidie-2,4-dione series and the substituted 5-[4-phenoxy-phenyl]-hydroxy-methyl -imidazolidine-2,4-dione series described in Example 8.

The key reaction is the aldol condensation (Method C) that forms the target compounds. The synthetic intermediates in this reaction are the 5-hydantoins, made from amino acids (Method A), and the aldehydes prepared through a Suzuki coupling (Method B) in a conventional manner. Method C also produces compounds 1. and 2. which may be utilized for further transformations, a Suzuki coupling (Method D) and amide coupling (Method E).

The aldol condensation gives a diastereomeric mixture. The racemates are isolated by chromatography or in some cases by crystallization. The enantiomeres may be resolved by chiral chromatography.

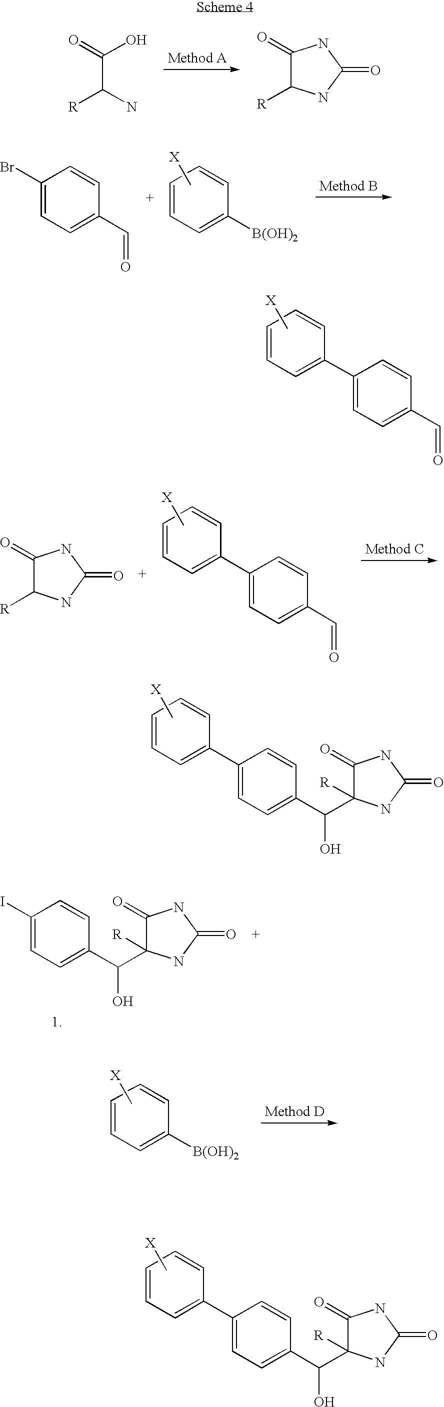

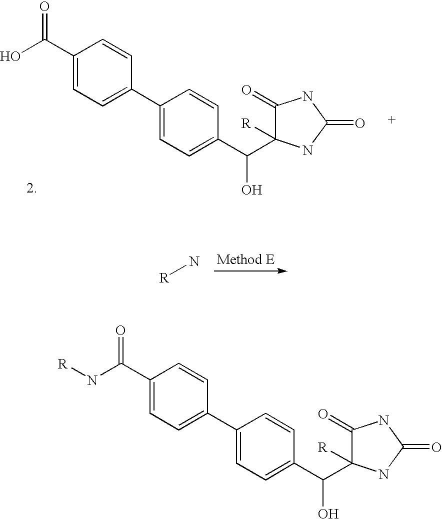

The compounds of the invention may be evaluated for example in the following assays:

Isolated Enzyme Assays

Matrix Metalloproteinase Family Including for Example MMP12, MMP13.

Recombinant human MMP12 catalytic domain may be expressed and purified as, described by Parkar A. A. et al, (2000), Protein Expression and Purification, 20:152. The purified enzyme can be used to monitor inhibitors of activity as follows: MMP12 (50 ng/ml final concentration) is incubated for 30 minutes at RT in assay buffer (0.1M Tris-HCl, pH 7.3 containing 0.1M NaCl, 20 mM CaCl₂, 0.040 mM ZnCl and 0.05% (w/v) Brij 35) using the synthetic substrate Mac-Pro-Cha-Gly-Nva-His-Ala-Dpa-NH2 in the presence or absence of inhibitors. Activity is determined by measuring the fluorescence at λex 328 nm and λem 393 nm. Percent inhibition is calculated as follows: % Inhibition is equal to the [Fluorescence_{plus inhibitor}-Fluorescence_{background}] divided by the [Fluorescence_{minus inhibitor}-Fluorescence_{background}].

Recombinant human proMMP13 may be expressed and purified as described by Knauper et al. [V. Knauper et al., (1996) The Biochemical Journal 271:1544-1550 (1996)]. The purified enzyme can be used to monitor inhibitors of activity as follows: purified proMMP13 is activated using 1 mM amino phenyl mercuric acid (APMA), 20 hours at 21° C.; the activated MMP13 (11.25 ng per assay) is incubated for 4-5 hours at 35° C. in assay buffer (0.1M Tris-HCl, pH 7.5 containing 0.1M NaCl, 20 mM CaCl2, 0.02 mM ZnCl and 0.05% (w/v) Brij 35) using the synthetic substrate 7-methoxycoumarin-4-yl)acetyl.Pro.Leu.Gly.Leu.N-3-(2,4-dinitrophenyl)-L-2,3-diaminopropionyl.Ala.Arg.NH₂ in the presence or absence of inhibitors. Activity is determined by measuring the fluorescence at λex 328 nm and λem 393 nm. Percent inhibition is calculated as follows: %

Inhibition is equal to the [Fluorescence_{plus inhibitor}-Fluorescence_{background}] divided by the [Fluorescence_{minus inhibitor}-Fluorescence_{background}].

A similar protocol can be used for other expressed and purified pro MMPs using substrates and buffers conditions optimal for the particular MMP, for instance as described in C. Graham Knight et al., (1992) FEBS Lett. 296(3):263-266.

Adamalysin Family Including for Example TNF Convertase

The ability of the compounds to inhibit proTNFα convertase enzyme may be assessed using a partially purified, isolated enzyme assay, the enzyme being obtained from the membranes of THP-1 as described by K. M. Mohler et al., (1994) Nature 370:218-220. The purified enzyme activity and inhibition thereof is determined by incubating the partially purified enzyme in the presence or absence of test compounds using the substrate 4′,5′-Dimethoxy-fluoresceinyl Ser.Pro.Leu.Ala.Gln.Ala.Val.Arg.Ser.Ser.Ser.Arg.Cys(4-(3-succinimid-1-yl)-fluorescein)-NH₂ in assay buffer (50 mM Tris HCl, pH 7.4 containing 0.1% (w/v) Triton X-100 and 2 mM CaCl₂), at 26° C. for 18 hours. The amount of inhibition is determined as for MMP13 except λex 490 nm and λem 530 nm were used. The substrate was synthesised as follows. The peptidic part of the substrate was assembled on Fmoc-NH-Rink-MBHA-polystyrene resin either manually or on an automated peptide synthesiser by standard methods involving the use of Fmoc-amino acids and O-benzotriazol-1-yl-N,N,N′,N′-tetramethyluronium hexafluorophosphate (HBTU) as coupling agent with at least a 4- or 5-fold excess of Fmoc-amino acid and HBTU. Ser¹ and Pro² were double-coupled. The following side chain protection strategy was employed; Ser¹(But), Gln⁵(Trityl), Arg^{8,12}(Pmc or Pbf), Ser^{9,10,11}(Trityl), Cys¹³(Trityl). Following assembly, the N-terminal Fmoc-protecting group was removed by treating the Fmoc-peptidyl-resin with in DMF. The amino-peptidyl-resin so obtained was acylated by treatment for 1.5-2 hr at 70° C. with 1.5-2 equivalents of 4′,5′-dimethoxy-fluorescein-4(5)-carboxylic acid [Khanna & Ullman, (1980) Anal Biochem. 108:156-161) which had been preactivated with diisopropylcarbodiimide and 1-hydroxybenzotiazole in DMF]. The dimethoxyfluoresceinyl-peptide was then simultaneously deprotected and cleaved from the resin by treatment with trifluoroacetic acid containing 5% each of water and triethylsilane. The dimethoxyfluoresceinyl-peptide was isolated by evaporation, trituration with diethyl ether and filtration. The isolated peptide was reacted with 4-(N-maleimido)-fluorescein in DMF containing diisopropylethylamine, the product purified by RP-HPLC and finally isolated by freeze-drying from aqueous acetic acid. The product was characterised by MALDI-TOF MS and amino acid analysis.

Natural Substrates

The activity of the compounds of the invention as inhibitors of aggrecan degradation may be assayed using methods for example based on the disclosures of E. C. Amer et al., (1998) Osteoarthritis and Cartilage 6:214-228; (1999) Journal of Biological Chemistry, 274 (10), 6594-6601 and the antibodies described therein. The potency of compounds to act as inhibitors against collagenases can be determined as described by T. Cawston and A. Barrett (1979) Anal. Biochem. 99:340-345.

Inhibition of Metalloproteinase Activity in Cell/tissue Based Activity

Test as an Agent to Inhibit Membrane Sheddases such as TNF Convertase

The ability of the compounds of this invention to inhibit the cellular processing of TNFα production may be assessed in THP-1 cells using an ELISA to detect released TNF essentially as described K. M. Mohler et al., (1994) Nature 370:218-220. In a similar fashion the processing or shedding of other membrane molecules such as those described in N. M. Hooper et al., (1997) Biochem. J. 321:265-279 may be tested using appropriate cell lines and with suitable antibodies to detect the shed protein.

Test as an Agent to Inhibit Cell Based Invasion

The ability of the compound of this invention to inhibit the migration of cells in an invasion assay may be determined as described in A. Albini et al., (1987) Cancer Research 47:3239-3245.

Test as an Agent to Inhibit Whole Blood TNF Sheddase Activity

The ability of the compounds of this invention to inhibit TNFα production is assessed in a human whole blood assay where LPS is used to stimulate the release of TNFα. Heparinized (10 Units/ml) human blood obtained from volunteers is diluted 1:5 with medium (RPMI1640+bicarbonate, penicillin, streptomycin and glutamine) and incubated (160 μl) with 20 μl of test compound (triplicates), in DMSO or appropriate vehicle, for 30 min at 37° C. in a humidified (5% CO₂/95% air) incubator, prior to addition of 20 μl LPS (E. coli. 0111:B4; final concentration 10 μg/ml). Each assay includes controls of diluted blood incubated with medium alone (6 wells/plate) or a known TNFα inhibitor as standard. The plates are then incubated for 6 hours at 37° C. (humidified incubator), centrifiged (2000 rpm for 10 min; 4° C.), plasma harvested (50-100μl) and stored in 96 well plates at −70° C. before subsequent analysis for TNFα concentration by ELISA.

Test as an Agent to Inhibit in vitro Cartilage Degradation

The ability of the compounds of this invention to inhibit the degradation of the aggrecan or collagen components of cartilage can be assessed essentially as described by K. M. Bottomley et al., (1997) Biochem J. 323:483-488.

Pharmacodynamic Test

To evaluate the clearance properties and bioavailability of the compounds of this invention an ex vivo pharmacodynamic test is employed which utilises the synthetic substrate assays above or alternatively HPLC or Mass spectrometric analysis. This is a generic test which can be used to estimate the clearance rate of compounds across a range of species. Animals (e,g. rats, marmosets) are dosed iv or po with a soluble formulation of compound (such as 20% w/v DMSO, 60% w/v PEG400) and at subsequent time points (e.g. 5, 15, 30, 60, 120, 240, 480,720, 1220 mins) the blood samples are taken from an appropriate vessel into 10U heparin. Plasma fractions are obtained following centrifugation and the plasma proteins precipitated with acetonitrile (80% w/v final concentration). After 30 mins at −20° C. the plasma proteins are sedimented by centrifugation and the supernatant fraction is evaporated to dryness using a Savant speed vac. The sediment is reconstituted in assay buffer and subsequently analysed for compound content using the synthetic substrate assay. Briefly, a compound concentration-response curve is constructed for the compound undergoing evaluation. Serial dilutions of the reconstituted plasma extracts are assessed for activity and the amount of compound present in the original plasma sample is calculated using the concentration-response curve taking into account the total plasma dilution factor.

In vivo Assessment

Test as an Anti-TNF Agent

The ability of the compounds of this. invention as ex vivo TNFα inhibitors is assessed in the rat. Briefly, groups of male Wistar Alderley Park (AP) rats (180-210 g) are dosed with compound (6 rats) or drug vehicle (10 rats) by the appropriate route e.g. peroral (p.o.), intraperitoneal (i.p.), subcutaneous (s.c.). Ninety minutes later rats are sacrificed using a rising concentration of CO₂ and bled out via the posterior vena cavae into 5 Units of sodium heparin/ml blood. Blood samples are immediately placed on ice and centrifuged at 2000 rpm for 10 min at 4° C. and the harvested plasmas frozen at −20° C. for subsequent assay of their effect on TNFα production by LPS-stimulated human blood. The rat plasma samples are thawed and 175 μl of each sample are added to a set format pattern in a 96U well plate. Fifty μl of heparinized human blood is then added to each well, mixed and the plate is incubated for 30 min at 37° C. (humidified incubator). LPS (25 μl; final concentration 10 μg/ml) is added to the wells and incubation continued for a further 5.5 hours. Control wells are incubated with 25 μl of medium alone. Plates are then centrifuged for 10 min at 2000 rpm and 200 μl of the supernatants are transferred to a 96 well plate and frozen at −20° C. for subsequent analysis of TNF concentration by ELISA.

Data analysis by dedicated software calculates for each compound/dose:

$$Percent inhibition of TNF α = \frac{Mean TNF α ( Controls ) - Mean TNF α ( Treated ) \times 100}{Mean TNF α ( Controls )}$$

Test as an Anti-arthritic Agent

Activity of a compound as an anti-arthritic is tested in the collagen-induced arthritis (CIA) as defined by D. E. Trentham et al., (1977) J. Exp. Med. 146,:857. In this model acid soluble native type II collagen causes polyarthritis in rats when administered in Freunds incomplete adjuvant. Similar conditions can be used to induce arthritis in mice and primates.

Test as an Anti-cancer Agent

Activity of a compound as an anti-cancer agent may be assessed essentially as described in I. J. Fidler (1978) Methods in Cancer Research 15:399-439, using for example the B16 cell line (described in B. Hibner et al., Abstract 283 p75 10th NCI-EORTC Symposium, Amsterdam Jun. 16-19 1998).

Test as an Anti-emphysema Agent

Activity of a compound as an anti-emphysema agent may be assessed essentially as described in Hautamaki et al (1997) Science, 277: 2002.

The invention will now be illustrated but not limited by the following Examples:

General analytical methods: ¹H-NMR spectra were recorded on either a Varian ^{Unity}Inova 400 MHz or Varian Mercury-VX300 MHz instrument. The central solvent peak of chloroform-d (δ_{H} 7.27 ppm), dimethylsulfoxide-d₆ (δ_{H} 2.50 ppm) or methanol-d₄ (δ_{H} 3.31 ppm) were used as internal references. Low resolution mass spectra were obtained on a Agilent 1100 LC-MS system equipped with an APCI ionization chamber.

If not stated otherwise commercially available starting materials or intermediates described in Table 2 and 3 where used.

### EXAMPLE 1

5-(Biphenyl-4-yl-hydroxy-methyl)-5-methyl-imidazolidine-2,4-dione

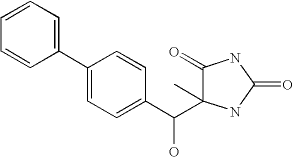

4-Biphenylcarboxaldehyde (182 mg, 1.0 mmol) and trimethylamine (45% in water, 160 μl, 1.0 mmol) was added to a warm solution of 5-methyl-imidazolidine-2,4, dione (114 mg, 1.0 mmol) in methanol (4.0 ml) and water (1.0 ml). The reaction was heated to reflux for 16 hours with nitrogen as inert atmosphere.

The solution was cooled, evaporated and stirred in a 100/1 mixture of dichloromethane/methanol (15 ml). Filtration, washing of the precipitate with the same solvent mixture (10 ml), and drying by airsuction, afforded 5-(Biphenyl-4-yl-hydroxy-methyl)-imidazolidine-2,4-dione (190 mg) in 64.1% yield as a diasteromeric mixture of 60/40 according to HNMR.

The isomeric mixture (180 mg) was dissolved in dioxane (8 ml) and water (4 ml). Preparative HPLC on a Chromasil C18 250/20 mm column (KR-100-5-C18), with a gradient of acetonitril/water (0.1% trifluoroacetic acid), from 20/80 to 40/60 during 25 min, afforded the two isolated diasteromeres in 43.5% total yield.

A preliminary stereostructural determination was done for each isomer by comparing the HNMR with the two diastereomeres of 5-[(4-chloro-phenyl)-hydroxy-methyl)]-imidazolidine-2,4-dione, of which both diasteromeric structures had been determined earlier by different NMR experiments in detail. The shift for the 1-NH proton and the phenyl attached to the imidazolelidione was especially indicative in this diastereomeric assignment.

(RR)-5-(Biphenyl-4-yl-hydroxy-(SS)-methyl)-5-methyl-imidazolidine-2,4-dione

1H NMR (400 MHz, DMSO-d6): 10.19 (1H, s); 8.11 (1H, s); 7.66 (2H, d, J=7.61 Hz); 7.59 (2H, d, J=8.20 Hz); 7.45 (2H, t, J=7.68 Hz); 7.37 (2H, d, J=8.27 Hz); 7.35 (1H, t, J=7.62 Hz); 5.92 (1H, bs); 4.67 (1H, s); 1.44 (3H, s).

13C NMR (400 MHz, DMSO-d6): 176.79; 156.25; 139.74; 139.39; 139.14; 128.91; 128.20; 127.37; 126.51; 125.54; 75.32; 66.96; 21.22.

APCI-MS m/z. 297.3 [MH+].

(SR)-5-(Biphenyl-4-yl-hydroxy-(RS)-methyl)-5-methyl-imidazolidine-2,4-dione

1H NMR (400 MHz, DMSO-d6): 10.48 (1H, s); 7.67 (2H, d, J=7.48 Hz); 7.64 (2H, d, J=8.29 Hz); 7.56 (1H, s); 7.48-7.45 (4H, m); 7.36 (1H, t, J=7.30 Hz); 5.75 (1H, d, J=4.73 Hz); 4.65 (1H, d, J=3.57 Hz); 1.08 (3H, s).

13C NMR (400 MHz, DMSO-d6): 177.89; 157.28; 139.88; 139.44; 139.27; 128.95; 128.47; 127.38; 126.54; 125.89; 74.68; 66.18; 20.22.

APCI-MS m/z: 297.3 [MH+].

The compounds described in Examples 2 to 4 were prepared using a method analogous to that given in Example 1.

### EXAMPLE 2

(RR)-5-(Biphenyl-4-yl-hydroxy-(SS)-methyl)-imidazolidine-2,4-dione

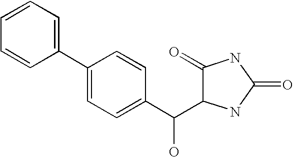

1H NMR (400 MHz, DMSO-d6): 10.33 (1H, s); 8.10 (1H, s); 7.66 (2H, d, J=8.20 Hz); 7.61 (2H, d, J=8.20 Hz); 7.45 (2H, dd, J=8.20/7.20 Hz); 7.39 (2H, d, J=8.24 Hz); 7.35 (1H, t, J=7.48 Hz); 5.89 (1H, bs); 4.97 (1H, d, J=2.5 Hz); 4.40 (1H, d, J=2.5 Hz).

APCI-MS m/z: 283.1 [MH+].

(SR)-5-(Biphenyl-4-yl-hydroxy-(RS)-methyl)-imidazolidine-2,4-dione

APCI-MS m/z: 283.1 [MH+].

### EXAMPLE 3

5-(Biphenyl-4-yl-hydroxy-methyl)-thiazolelidine-2,4-dione

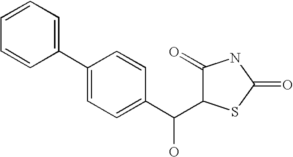

(RR)-5-(Biphenyl-4-yl-hydroxy-(SS)-methyl)-thiazolelidine-2,4-dione

1H NMR (400 MHz, DMSO-d6): 11.81 (1H, s); 7.68 (2H, d, J=8.20 Hz); 7.64 (2H, d, J=8.20 Hz); 7.46 (2H, dd, J=8.30/7.50 Hz); 7.42 (2H, d, J=8.30 Hz); 7.36 (1H, t, J=7.50 Hz); 6.24 (1H, d, J=3.96 Hz); 5.36 (1H, t, J=3.95 Hz); 5.06 (1H, d, J=4.03 Hz).

APCI-MS m/z: 183.1 [MH+-thiazolelidine-2,4-dione].

(SR)-5-(Biphenyl-4-yl-hydroxy-(RS)-methyl)-thiazolelidine-2,4-dione

1H NMR (400 MHz, DMSO-d6): 12.04 (1H, s); 7.67 (2H, d, J=8.30 Hz); 7.65 (2H, d, J=8.30 Hz); 7.51 (2H, d, J=8.20 Hz); 7.46 (2H, dd, J=8.20/7.40 Hz); 7.36 (1H, t, J=7.40 Hz); 6.22 (1H, d, J=5.20 Hz); 5.42 (1H, dd, J=5.20/2.60 Hz); 5.02 (1H, d, J 2.60 Hz).

APCI-MS m/z: 183.1 [MH+-thiazolelidine-2,4-dione].

### EXAMPLE 4

5-(Biphenyl-4-yl-hydroxy-methyl)-1-methyl-imidazolidine-2,4-dione

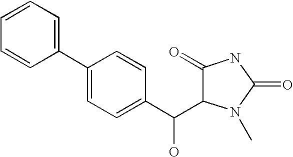

(RR)-5(Biphenyl-4-yl-hydroxy-(SS)-methyl)-1-methyl-imidazolidine-2,4-dione

1H NMR (400 MHz, DMSO-d6): 10.53 (1H, s); 7.67 (2H, d, J=7.20 Hz); 7.63 (2H, d, J=8.43 Hz); 7.46 (2H, dd, J=7.71/7.20 Hz); 7.38 (2H, d, J=8.63 Hz); 7.35 (1H, t, J=7.63 Hz); 6.01(1H, d, J=4.16 Hz); 5.13 (1H, dd, J=4.18/2.60 Hz); 4.33 (1H, d, J=2.58 Hz); 2.97 (3H, s).

13C NMR (400 MHz, DMSO-d6): 176.63; 156.83; 139.78; 138.97; 138.95; 128.89; 127.35; 127.13; 126.53; 125.91; 71.28; 67.81; 28.63.

APCI-MS m/z: 297.1 [MH+]

(SR)-5-(Biphenyl-4-yl-hydroxy-(RS)-methyl)-1-methyl-imidazolidine-2,4-dione

1H NMR (460 MHz, DMSO-d6): 10.73 (1H, s); 7.70 (4H, m); 7.54 (2H, d, J=8.22 Hz); 7.46 (2H, dd, J=8.20/7.10 Hz); 7.36.(1H, t, J=7.11 Hz); 5.96 (1H, d, J=6.06 Hz); 5.11 (1H, dd, J=6.06/2.14 Hz); 4.38 (1H, d, J=2.14 Hz); 2.33 (3H, s).

APCI-MS m/z: 297.1 [MH+]

### EXAMPLE 5

5-[Hydroxy-(3-phenoxy-phenyl)-methyl]-imidazolidine-2,4-dione

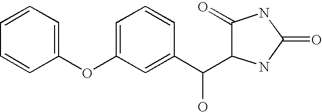

The compound was prepared according to the method given in Example 1 but instead of preparation by HPLC, flash chromatography (SiO, dichloromethane/methanol: gradient to 100/4) afforded 60 mg of the title compound as a white solid in 20.1% yield (diastereomeric mixture). HNMR confirmed that the ratio of the mixture of the diastereomeric isomers was 1:1.

1H NMR (400 MHz, DMSO-d6): 10.51 (1H, bs); 10.37 (1H, bs); 8.04 (1H, s); 7.56 (1H, s); 7.40-7.29 (6H, m); 7.16-7.09 (4H, m); 7.05-7.02 (4H, m); 6.96 (2H, d, J=8.71 Hz); 6.89 (2H, m); 5.89 (1H, d, J=3.91 Hz); 5.78 (1H, d, J 5.68 Hz); 4.93-4.90 (2H, m); 4.34 (1H,dd); 4.25 (1H, dd).

13C NMR (400 MHz, DMSO-d6): 174.04; 173.05; 158.09; 157.40; 156.89; 156.83; 156.31; 155.63; 144.01; 141.69; 129.96; 129.94; 129.55; 129.15; 123.20; 123.06; 122.26; 121.28; 118.44; 118.06; 118.02; 117.80; 117.46; 116.76; 71.98; 70.28; 64.01.

APCI-MS m/z: 281.1 [MH+−H2O].

### EXAMPLE 6

5-[Hydroxy-(4-phenoxy-phenyl)-methyl]-imidazolidine-2,4-dione

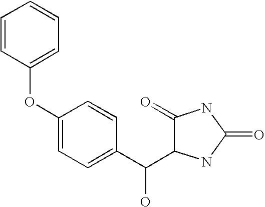

The compound was prepared according to the method given in Example 1 but instead of preparation by HPLC, flash chromatography (SiO, dichloromethane/methanol: gradient to 100/3) afforded 40 mg of the title compound as a white solid in 13.4% yield (diastereomeric mixture). HNMR confirmed that the ratio of the mixture of the diastereomeric isomers was 1:1.

1H NMR (400 MHz, DMSO-d6): 10.49 (1H, bs); 10.36 (1H, bs); 8.04 (1H, s); 7.55 (1H, s); 7.41-7.35 (6H, m); 7.31 (2H, d, J=8.60 Hz); 7.13 (2H, ddd, J=7.44/3.52/1.14 Hz); 7.01-6.92 (8H, m); 5.84 (1H, d, J=3.76 Hz); 5.74 (1H, d, J=5.55 Hz); 4.91 (2H, m); 4.34 (1H,dd, J=3.03/1.05 Hz); 4.22 (1H, DD, 2.68/1.52 Hz).

APCI-MS m/z: 281.1 [MH+-H2O].

### EXAMPLE 7

The following compounds were made according to the methods described for the Examples above.

5-[(4′-Fluoro-biphenyl-4-yl)-hydroxy-methyl]-imidazolidine-2,4-dione

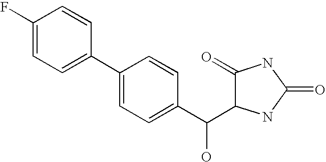

APCI-MS m/z: 283 [MH+-H2O].

5-[(4′-Fluoro-biphenyl-4-yl)-hydroxy-methyl]-5-methyl-imidazolidine-2,4-dione

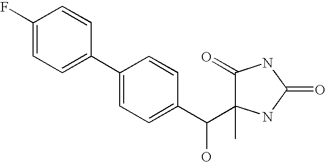

APCI-MS m/z: 314.9 [MH+].

5-[(4′-Fluoro-biphenyl-4-yl)-hydroxy-methyl]-5-isobutyl-imidazolidine-2,4-dione

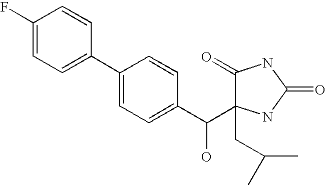

APCI-MS m/z: 357.1 [MH].

5-[(4′-Chloro-biphenyl-4-yl)-hydroxy-methyl]-imidazolidine-2,4-dione

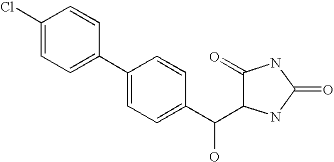

APCI-MS m/z: 298.9 [MH+-H2O].

5-[(4′-Chloro-biphenyl-4-yl)-hydroxy-methyl]-5-methyl-imidazolidine-2,4-dione

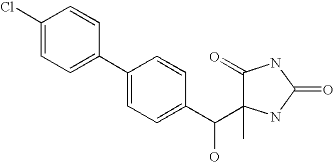

APCI-MS m/z: 331 [MH+].

5-[(4′-Chloro-biphenyl-4-yl)-hydroxy-methyl]-5-isobutyl-imidazolidine-2,4-dione

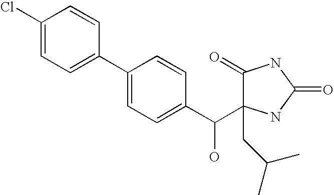

APCI-MS m/z: 373.1 [MH+].

5-(Biphenyl-4-yl)-hydroxy-methyl]-5-hydroxymethyl-imidazolidine-2,4-dione

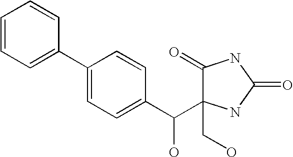

APCI-MS m/z: 313.0 [MH+].

### EXAMPLE 8

Compounds were synthesized according to Method C in Scheme 4 (shown in the description above).

(a) Preparation of Intermediate Hydantoins (Method A in Scheme 4)

According to Scheme 5 below, the hydantoins 5 were prepared in two steps from general amino acids 3 with isolation of the intermediates 4.

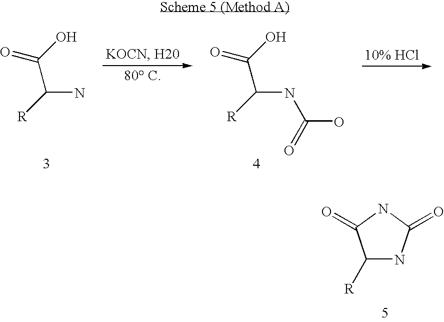

Table 2 lists the intermediate hydantoins that were synthesized. The general method of preparation was as follows. A slurry of amino acid 3 (25 mmol) and potassium cyanate (5.1 g, 63 mmol) in water (75 ml) was heated at 80° C. for approximately 1 hour. The clear solution was cooled to 0° C. and acidified to approximately pH 1 with concentrated hydrochloric acid (aq). The resulting white precipitate 4 was heated at reflux for 0.5-1 hour and then cooled on ice. In some instances full conversion was not reached after 1 hour heating. In these cases the crude material was treated under the same protocol again. The white solid was filtered, washed with water, dried and analysed by HNMR and LCMS.

TABLE 2
intermediate hydantoins

| | | APCI- |
|---|---|---|
| | | MS |
| | Yield | m/z: |
| Name: | (%) | [MH⁺] |
| 5-(4-Chloro-benzyl)-imidazolidine-2,4-dione | 87 | 224.9 |
| [3-(2,5-Dioxo-imidazolidin-4-yl)-propyl]- | 50 | 292.0 |
| carbamic acid benzyl ester | | |
| 5-Isobutyl-imidazolidine-2,4-dione | 85 | 157.0 |
| 5-Benzylsulfanylmethyl-imidazolidine-2,4-dione | 87 | 237.0 |
| 5-Methylsulfanylmethyl-imidazolidine-2,4-dione | 45 | 161.0 |
| 5-Cyclohexylmethyl-imidazolidine-2,4-dione | 63 | 197.1 |
| 5-sec-Butyl-imidazolidine-2,4-dione | 52 | 157.0 |
| 5-Phenethyl-imidazolidine-2,4-dione | 94 | 205.1 |
| 5-Butyl-imidazolidine-2,4-dione | 82 | 157.0 |
| 5-Isopropyl-imidazolidine-2,4-dione | 49 | |
| 5-(1H5-Indol-3-ylmethyl)-imidazolidine-2,4-dione | 94 | 230.0 |
| 5-(2-Hydroxy-ethyl)-imidazolidine-2,4-dione | 36 | |

(b) Preparation of Intermediate Aldehydes (Method B in Scheme 4)

Substituted benzaldehydes where prepared by Suzuki coupling between different commercially available phenyl bromides and 4-formylphenylboronic acid, according to Scheme 6 below.

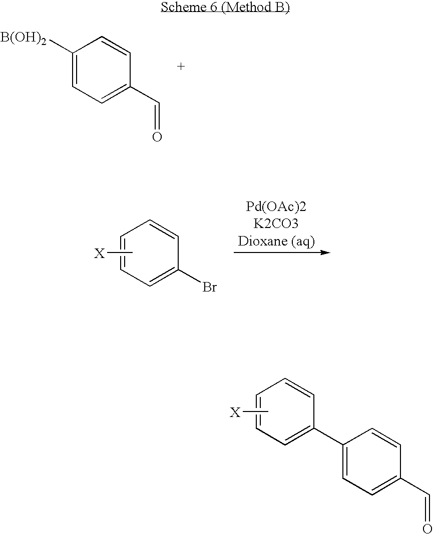

4-pyridin-2-yl-benzaldehyde

The compound was prepared as follows. A mixture of 4-formylphenylboronic acid (195 mg,1.3 mmol), 2-bromopyridine (102.7 mg, 0.65mmol) and powdered K₂CO₃ (1.07 g, 5 7.8 mmol) in dioxane (12 ml) and water (2 ml) was deoxygenated (vacuum and argon). Palladium diacetate (30 mg, 0.2 mol %) was added and the mixture was stirred for 2 hours at 80° C. under argon.

The slurry was cooled to room temperature. Filtration and evaporation afforded the crude product. Preparative HPLC (Chromasil C18 column, acetonitrile, water and trifluoroacetic acid), afforded the title compound 4-pyridin-2-yl-benzaldehyde (72 mg, in 60% yield.

¹H NMR(400 MHz, DMSO-d6): δ10.07 (1H, s); 8.73 (1H, d, J=4.20 Hz); 8.31 (2H, d, J=8.20); 8.11 (1H, d, J=8.01); 8.03 (2H, d, J 8.20); 7.97 (1H, m). APCI-MS m/z: 184.2 [MH+].

Other substituted benzaldehydes (listed in Table 3) were produced according to the same method.

TABLE 3
Substituted benzaldehydes

| | | APCI- |
|---|---|---|
| Name: | Yield (%) | MS m/z: |
| 4′-Formyl-biphenyl-4-carbonitrile | 65 | 208.0 |
| 4′-Formyl-biphenyl-3-carbonitrile | | 208.0 |
| 4′-Methoxy-biphenyl-4-carbaldehyde | 50 | 213.1 |
| 3-Methoxy-biphenyl-4-carbaldehyde | 62 | 213.1 |
| Biphenyl-4,4′-dicarbaldehyde | | 211.0 |
| Acetic acid 4′-formyl-biphenyl-3-yl ester | | 239.1 |
| Acetic acid 4′-formyl-biphenyl-4yl ester | | 239.1 |
| N-(4′-Formyl-biphenyl-3-yl)-acetamide | 75 | 240.1 |
| 4′-Hydroxymethyl-biphenyl-4-carbaldehyde | 55 | 213.1 |
| 3′-Fluoro-biphenyl-4-carbaldehyde | 70 | 201.1 |
| 4-Pyridine-3-yl-benzaldehyde | 67 | 184.2 |
| 3′,4′-Difluoro-biphenyl-4-carbaldehyde | 72 | 219.1 |
| 4-Pyridine-4-yl-benzaldehyde | 67 | 184.2 |
| N-[4-(4-Formyl-phenyl)-pyridine-2-yl]-acetamide | 30 | 241.0 |
| 4-Benzo[1,3]dioxol-5-yl-benzaldehyde | 20 | 226.1 |

(c) Aldol Condensation of Intermediate Hydantoins and Aldehydes (Method C in Scheme 4)

The general procedure is exemplified by the synthesis of 5-{[4-(4-Fluoro-phenoxy)-phenyl]-methyl-methyl}-5-propyl-imidazolidine-2,4-dione below.

5-{[4-(4-Fluoro-phenoxy)-phenyl]-methyl-methyl}-5-propyl-imidazolidine-2,4-dione

Commercially available 4-(4-fluoro-phenoxy)-benzaldehyde (201.5 mg, 1.0 mmol), 5-propyl-hydantoin (438mg, 3.08 mmol) and 45% aqueous trimethylamine (0.240 ml, 1.5 mmol) was refluxed in ethanol (12 ml) and water (3 ml) for. 20 hours.

Evaporation and preparative HPLC( C18 column, acetonitrile, water and trifluoro acetic acid) afforded the title compound 5-{[4-(4-Fluoro-phenoxy)-phenyl]-methyl-methyl}-5-propyl-imidazolidine-2,4-dione (11 mg, 0.03 mmol) in 3% yield as white solid in form of the pure racemate.

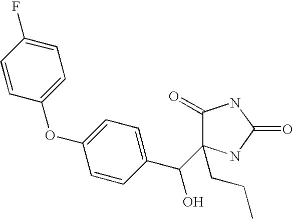

¹HNMR (300 MHz, DMSO-₆): δ10.71 (1H, s); 7.99 (1H, s); 7.70 (2H, dd, J=4.38, 5.37 Hz); 7.75 (2H, d, J=8.44 Hz); 7.35 (2H, d, J=8.03 Hz); 7.27 (2H, dd, J=4.59, 8.60 Hz); 5.89 (1H, d, J=4.42 Hz); 4.66 (1H, d, J=4.34 Hz); 1.96 (1H, dd, J=12.89, 4.36 Hz); 1.71 (1H, dd; J=12.95, 4.77 Hz); 1.32 (1H, m); 1.10 (1H, m); 0.89 (3H, t, J=7.49 Hz).

APCI-MS m/z: 343.1 [MH⁺-OH].

The following compounds were produced according to the same method. 5-[4-phenoxy-phenyl]-hydroxy-methyl]-5-methyl-imidazolidine-2,4-dione

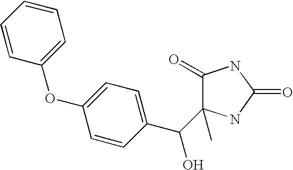

¹HNMR (400 MHz, DMSO-d₆): δ10.12 (1H, bs); 8.06 (1H, s); 7.38 (2H, dd, J=3.94, 7.60 Hz); 7.28 (2H, d, J=8.62 Hz); 7.13 (1H, t, J=7.43 Hz); 6.96 (2H, d, J=8.75 Hz); 6.91 (2H, d, J=8.61 Hz); 5.89 (1H, d, J=4.33 Hz); 4.62 (1H, d, J=4.48 Hz); 1.41 (3H, s).

APCI-MS m/z: 313.0 [MH⁺]

4-[Hydroxy-(4-methyl-2,5-dioxo-imidazolidine 4-yl)-methyl]-piperidine-1-carboxylic acid benzyl ester.

Prepared from commercially available starting materials.

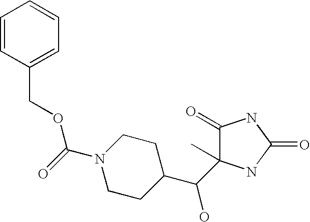

APCI-MS m/z: 362.1[MH⁺].

5-[(4′-Fluoro-biphenyl-4-yl)-hydroxy-methyl]-imidazolidine-2,4-dione

Prepared from commercially available starting materials.

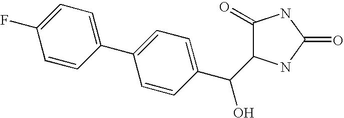

¹HNMR (400 MHz, DMSO-d₆): δ10.32 (1H, s); 8.09 (1H, s); 7.71 (2H, dd, J=4.47, 5.60 Hz); 7.60 (2H, d, J=8.27 Hz); 7.38 (2H, d, J=8.33 Hz); 7.28 (2H, dd, J=5.05, 8.68 Hz); 5.88 (1H, d, J=3.90 Hz); 4.97 (1H, t, J=3.29 Hz); 4.39 (1H, d, J=2.64 Hz).

APCI-MS m/z: 301.2 [MH⁺].

5-Ethyl-5-[(41-fluoro-biphenyl-4-yl)-hydroxy-methyl]-imidazolidine-2,4-dione

Produed by aldol condensation of 4′-fluoro-biphenyl-4carbaldehyde and 5-Ethyl-imidazolide-2,4-dione.

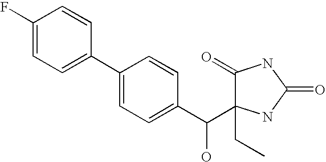

1H NMR (400 MHz, DMSO-d6): δ10.18 (1H, s); 7.96 (1H, s); 7.69 (2H, dd, J=8.77/5.53 Hz); 7.57 (2H, d, J=8.2 Hz); 7.35 (2H, d, J=8.20 Hz); 7.26 (2H, t, J=8.87 Hz) 5.87 (1H, d, J=4.39 Hz); 4.66 (1H, d, 4.39 Hz); 1.98 (1H, m); 1.75 (1H, m); 0.78 (3H, t, J=7.34 Hz).

APCI-MS m/z: 329.1 [MH⁺]

5-[(4′-fluoro-biphenyl-4-yl)-hydroxy-methyl]-5-propyl-imidazolidine-2,4-dione

Produced by aldol condensation of 4′-fluoro-biphenyl-4-carbaldehyde and 5-propyl-imidazolidine-2,4-dione.

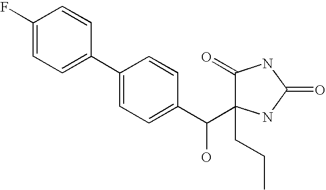

1H NMR (400 MHz, DMSO-d6): δ10.16 (1H, s); 7.98 (1H, s); 7.69 (2H, dd, J=8.68/5.44 Hz); 7.56 (2H, d, J=8.20 Hz); 7.34 (2H, d, J=8.20 Hz); 7.26 (2H, t, J=8.77 Hz) 5.87 (1H, d, J=4.39 Hz); 4.64 (1H, d, 4.39 Hz); 1.94 (1H, m); 1.70 (1H, m); 1.31 (1H, m); 1.10 (1H, m); 0.88 (3H, t, J=7.34 Hz).

APCI-MS m/z: 343.1 [MH⁺]

5-[Hydroxy-(4′-methoxy-biphenyl-4-yl)-methyl]-5-methyl-imidazolidine-2,4-dione Produced by aldol condensation of 4′-Methoxy-biphenyl-4-carbaldehyde and 5-Methyl-imidazolidine-2,4-dione.

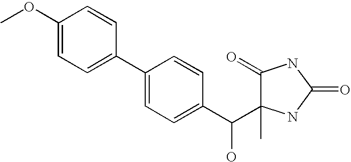

1H NMR (400 MHz, DMSO-d6): δ10.16 (1H, s); 8.08 (1H, s); 7.59 (2H, d, J=8.77 Hz); 7.52 (2H, d, J=8.20 Hz); 7.31 (2H, d, J=8.20 Hz); 6.99 (2H, d, J=8.58 Hz); 5.87 (1H, d, J=4.39 Hz); 4.63 (1H, d, 4.39 Hz); 3.77 (3H, t); 1.42 (3H, s).

APCI-MS m/z: 327.1 [MH⁺]

5-[Hydroxy-(3′-methoxy-biphenyl-4-yl)-methyl]-5-methyl-imidazolidine-2,4-dione

Produced by aldol condensation of 3-Methoxy-biphenyl-4-carbaldehyde and 5-Methyl-imidazolidine-2,4-dione.

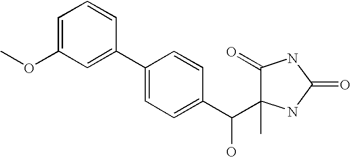

1H NMR (400 MHz, DMSO-d6): δ10.18 (1H, s); 8.08 (1H, s); 7.59 (2H, d, J=8.01 Hz); 7.35 (3H, m); 7.21 (1H, d, J=7.63 Hz); 7.17 (1H, s); 6.91 (1H, dd, J=8.11/2.19) 5.91 (1H, d, J=4.39 Hz); 4.65 (1H, d, 4.39 Hz); 3.81 (3H, t); 1.43 (3H, s).

APCI-MS m/z: 327.1 [MH⁺]

4′-[Hydroxy-(4-methyl-2,5-dioxo-imidazolidinyl)-methyl]-biphenyl-4-carbonitrile

Produced by aldol condensation of 4′-Formyl-biphenyl-4-carbonitrile and 5-Methyl-imidazolidine-2,4-dione.

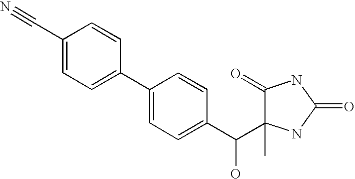

1H NMR (400 MHz, DMSO-d6): δ10.18 (1H, s); 8.11 (1H, s); 7.89 (4H, m); 7.69 (2H, d, J=8.20); 7.40 (2H, d, J=8.20 Hz); 5.97 (1H, d, J=4.39 Hz); 4.67 (1H, d, 4.39 Hz); 3.81 (3 H, t); 1.43 (3H, s).

APCI-MS m/z: 322.1 [MH⁺]

4′-[-Hydroxy-(4-methyl-2,5-dioxo-imidazolidin-4-yl)-methyl]-biphenyl-3-carbonitrile

Produced by aldol condensation of 4′-Formyl-biphenyl-3-carbonitrile and 5-Methyl-imidazolidine-2,4-dione.

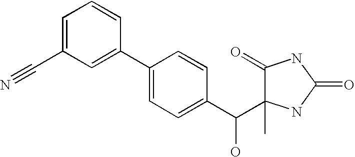

1H NMR (400 MHz, DMSO-d6): δ10.18 (1H, s); 8.14 (1H, s); 8.11 (1H,s); 8.02 (1H, d, J=8.01 Hz); 7.80 (1H, d, J=7.63 Hz); 7.69 (2H, d, J=8.20 Hz); 7.64 (1H, t, J=7.82 Hz); 7.38 (2H, d, J=8.20 Hz); 5.96 (1H, d, J=4.20 Hz); 4.67 (1H, d, 3.81 Hz); 1.42 (3H, s)

APCI-MS m/z: 322.1 [MH⁺]

4′-[Hydroxy-(4-methyl-2,5-dioxo-imidazolidin-4-yl)-methyl]-biphenyl-4-carbaldehyde

Produced by aldol condensation of biphenyl-4-4′-dicarbaldehyde and 5-Methyl-imidazolidine-2,4-dione.

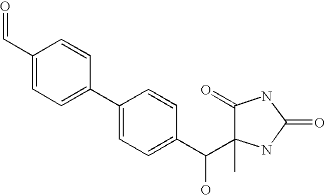

1H NMR (400 MHz, DMSO-d6): δ10.19 (1H, s); 10.03 (1H, s); 8.12 (1H, s); 7.97 (2H, d, J=8.40 Hz); 7.91 (2H, d, J=8.40); 7.71 (2H, d, J=8.20 Hz); 7.40 (2H, d, J=8.40 Hz); 5.97 (1H, d, J=4.39 Hz); 4.67 (1H, d, 4.39 Hz); 3.81 (3H, t); 1.43 (3H, s).

APCI-MS m/z: 325.1 [MH⁺]

Acetic acid 4′-[hydroxy-(4-methyl-2,5-dioxo-imidazolidin-4-yl)-methyl]-biphenyl-3-yl-ester

Produced by aldol condensation of acetic acid 4′-formyl-biphenyl-3-yl ester and 5-Methyl-imidazolidine-2,4-dione.

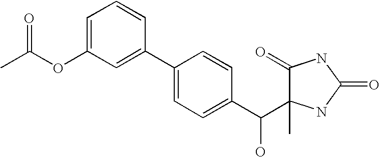

1H NMR (400 MHz, DMSO-d6): δ10.18 (1H, s); 8.16 (1H, s); 8.11 (1H, s); 7.92 (1H, dd, J=7.72/1.24 Hz), 7.66 (2H, d, J=8.40); 7.60 (1H, t, J=7.73 Hz); 7.38 (2H, d, J=8.40 Hz); 5.94 (1H, d, J=4.39 Hz); 4.67 (1H, d, 4.39 Hz); 2.63 (3H, s); 1.42 (3H, s).

APCI-MS m/z: 321.1 [MH⁺-H₂O]

Acetic acid 4′-[hydroxy-(4-methyl-2,5-dioxo-imidazolidin-4-yl)-methyl]-biphenyl-4-yl-ester

Produced by aldol condensation of acetic acid 4′-formyl-biphenyl-4-yl ester and 5-Methyl-imidazolidine-2,4-dione.

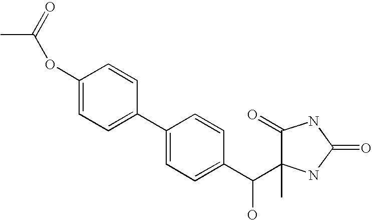

1H NMR (400 MHz, DMSO-d6): δ10.19 (1H, s); 8.11 (1H, s); 8.01 (2H, d, J=8.39 Hz) 7.82 (2H, d, J=8.20); 7.68 (2H, d, J=8.20 Hz); 7.39 (2H, d, J=8.20 Hz); 5.96 (1H, d, J=4.39 Hz); 4.67 (1H, d, 4.39 Hz); 2.59 (3H, t); 1.43 (3H, s).

APCI-MS m/z: 321.1 [MH⁺-H₂O]

N-{4′-[Hydroxy-(4-methyl-2,5-dioxo-imidazolidin-4-yl)-methyl]-biphenyl-3-yl}-acetamide

Produced by aldol condensation of N-(4′-Formyl-biphenyl-3-yl)-acetamide and 5-Methyl-imidazolidine-2,4-dione.

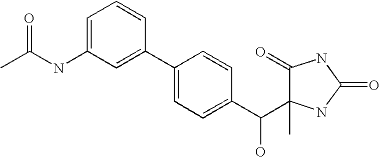

1H NMR (400 MHz, DMSO-d6): δ10.17 (1H, s); 9.98 (1H, s); 8.08 (1H, s); 7.87 (1H, s); 7.50 (3H, m); 7.32 (4H, m); 5.91 (1H, d, J=4.56 Hz); 4.64 (1H, d, 4.28 Hz); 2.05 (3H, s); 1.42 (3H, s).

APCI-MS m/z: 354:1 [MH⁺]

5-[Hydroxy-(4-hydroxymethyl-biphenyl-4-yl)-methyl]-5-methyl-imidazolidine-2,4-dione

Produced by aldol condensation of 4′-Hydroxymethyl-biphenyl-4-carbaldehyde and 5-Methyl-imidazolidine-2,4-dione.

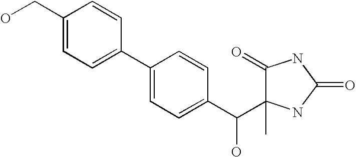

1H NMR (400 MHz, DMSO-d6): δ10.17 (1H, s); 8.09 (1H, s); 7.61 (2H, d, J=8.20 Hz) 7.57 (2H, d, J=8.20); 7.38 (2H, d, J=8.20 Hz); 7.34 (2H, d, J=8.20 Hz); 5.90 (1H, d, J=4.39 Hz); 5.19 (1H, T, J=5.72 Hz); 4.65 (1H, d, 4.39 Hz); 4.52 (2H, d, J=5.72 Hz); (3H, s).

APCI-MS m/z: 327.1 [MH⁺]

5-[(4-Benzyloxy-phenyl)-hydroxy-methyl]-5-methyl-imidazolidine-2,4-dione

Produced by aldol condensation of 4-benzyloxy-benzaldehyde and 5-Methyl-imidazolidine-2,4-dione.

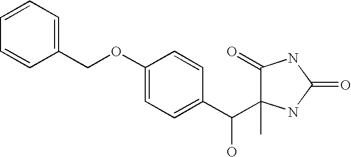

1H NMR (400 MHz, DMSO-d6): δ10.10 (1H, s); 8.01 (1H, s); 7.46-7.27 (5H, m); 7.18 (2H, d, J=8.58 Hz); 6.89 (2H, d, J=8.58 Hz); 5.75 (1H, d, J=4.39 Hz); 5.04 (2H, s); 4.55 (1H, d, J=4.39 Hz); 1.43 (3H, s).

APCI-MS m/z: 309.1 [MH⁺-H₂O]

5-[Hydroxy-(4-pyridine-3-yl-phenyl)-methyl]-5-methyl-imidazolidine-2,4-dione

Produced by aldol condensation of 4-Pyridine-3-yl-benzaldehyde and 5-Methyl-imidazolidine-2,4-dione.

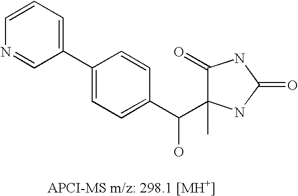

5-[(3′-Fluoro-biphenylyl)-hydroxy-methyl]-5-methyl-imidazolidine-2,4-dione

Produced by aldol condensation of 3′-Fluoro-biphenyl-4-carbaldehyde and 5-Methyl-imidazolidine-2,4-dione.

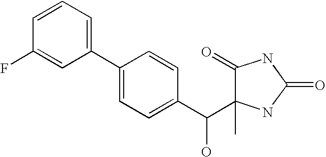

1H NMR (400 MHz, DMSO-d6): δ10.17 (1H, s); 8.10 (1H, s); 7.63 (1H, d, J=8.20 Hz); 7.49 (3H, m); 7.36 (2H, d, J=8.20 Hz); 7.17 (1H, m); 5.93 (1H, d, J=4.20 Hz); 4.66 (1H, d, 3.81 Hz); 1.42 (3H, s).

APCI-MS m/z: 315 [MH⁺]

5-[Hydroxy-(4-phenylethenyl-phenyl)-methyl]-5-methyl-imidazolidine-2,4-dione

The starting aldehyde was synthesized according; Thorand S. et.al (J Org Chem 1998, 63(23), 8551-8553).

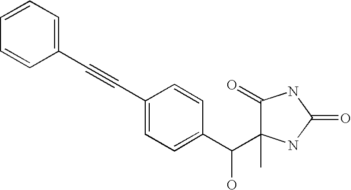

1H NMR (400 MHz, DMSO-d6): δ10.18 (1H, s); 8.08 (1H, s); 7.53 (2H, m); 7.45 (2H, d, J=8.40 Hz); 7.41 (3H, m); 7.30 (2H, d, J=8.20 Hz); 5.99 (1H, d, J=4.58 Hz); 4.64 (1H, d, 4.39 Hz); 1.41 (3H, s).

APCI-MS m/z: 321.1 [MH⁺]

5-[Hydroxy-(4-pyridine-4-yl-phenyl)-methyl]-5-methyl-imidazolidine-2,4-dione

Produced by aldol condensation of 4-Pyridine-4-yl-benzaldehyde and 5-Methyl-imidazolidine-2,4-dione.

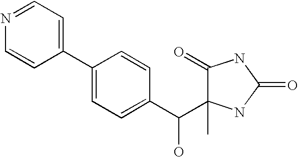

1H NMR (400 MHz, DMSO-d6): δ10.19 (1H, s); 8.61 (2H, m); 8.12 (1H, s); 7.74 (2H, d, J=8.39); 7.70 (2H, m); 7.41 (2H, d, J=8.20 Hz); 5.99 (1H, s,); 4.67 (1H, s); 1.42 (3H, s).

APCI-MS m/z: 298.1 [MH⁺]

N-{4′-[Hydroxy-(4-methyl-2,5-dioxo-imidazolidin-4-yl)-methyl]-biphenyl-4-yl)-acetamide

Produced by aldol condensation of N-(4′-formyl-biphenyl-4-yl)-acetamide and 5-Methyl-imidazolidine-2,4-dione.

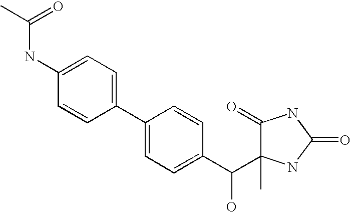

APCI-MS m/z: 354.1 [MH⁺]

N-(5-{4-[Hydroxy-(4-methyl-2,5-dioxo-imidazolidin-4-yl)-methyl]-phenyl}-pyridin-2-yl)-acetamide

Produced by aldol condensation of N-[4-(4-Formyl-phenyl)-pyridine-2-yl]-acetamide and 5-Methyl-imidazolidine-2,4-dione.

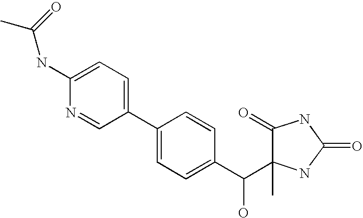

APCI-MS m/z: 355.1 [MH⁺]

5-[(3′,4′-Difluoro-biphenyl-4-yl)-hydroxy-methyl]-5-methyl-imidazolidine-2,4-dione

Produced by aldol condensation of 3′,4′-Difluoro-biphenyl-4-carbaldehyde and 5-Methyl-imidazolidine-2,4-dione.

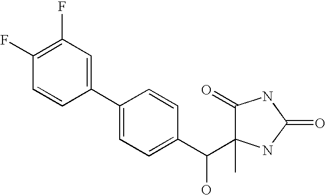

1H NMR (400 MHz, DMSO-d6): δ10.16 (1H, s); 8.10 (1H, s); 7.75 (1H, m); 7.61 (2H, d, J=8.27 Hz); 7.50 (2H, m); 7.35 (2H, d, J=8.27); 5.93 (1H, d, J=3.99 Hz); 4.66 (1H, d, 3.98 Hz); 1.41 (3H, s).

APCI-MS m/z: 333 [MH⁺]

5-[Hydroxy-(4-[1,2,3]thiadiazol-5-yl-phenyl)-methyl]-5-methyl-imidazolidine-2,4-dione

Produced by aldol condensation of 4-[1,2,3]Thiadiazol-5-yl-benzaldehyde and 5-Methyl-imidazolidine-2,4-dione.

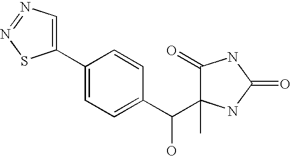

APCI-MS m/z: 305 [MH⁺]

5-{[5-(2-Chloro-4-trifluoromethyl-phenyl)-furan-2-yl]-hydroxy-methyl}-5-methyl-imidazolidine-2,4-dione

Produced by aldol condensation of 5-(3-chloro-4-trifluoromethyl-phenyl)-furan-2-carbaldehyde and 5-Methyl-imidazolidine-2,4-dione.

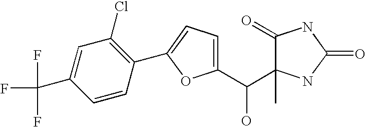

APCI-MS m/z: 389 [MH⁺]

5-{[5-(4-Chloro-phenylsulfinyl)-thiophene-2-yl]-hydroxy-methyl)-5-methyl-imidazolidine-2,4-dione

Produced by aldol condensation of 5-(4-chloro-phenylsulfanyl)-thiophene-2-carbaldehyde and 5-Methyl-imidazolidine-2,4-dione.

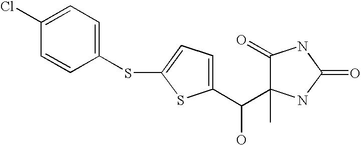

APCI-MS m/z: 350.9 [MH⁺-H₂O]

5-{[4-(4-tert-Butyl-thiazol-2-yl)-phenyl]-hydroxy-methyl}-5-methyl-imidazolidine-2,4-dione

Produced by aldol condensation of 4-(4-tert-butyl-thiazol-2-yl)-benzaldehyde and 5-Methyl-imidazolidine-2,4-dione.

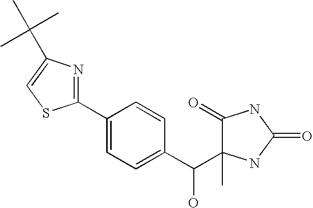

APCI-MS m/z: 360 [MH⁺]

5-{[4-(2-Chloro-6-fluoro-benzyloxy)-3-methoxy-phenyl]-hydroxy-methyl}-5-methyl-imidazolidine-2,4-dione

Produced by aldol condensation of 4-(2-chloro-6-fluoro-benzyloxy)-3-methoxy-bemnzaldehyde and 5-Methyl-imidazolidine-2,4-dione.

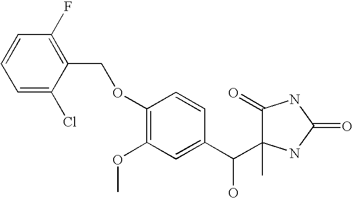

APCI-MS m/z: 391 [MH⁺-H₂O]

5-{[2-4-Chloro-phenylsulfanyl)-phenyl]-hydroxy-methyl}-5-methyl-imidazolidine-2,4-dione

Produced by aldol condensation of 2-(4-chloro-phenylsulfanyl)-benzaldehyde and 5-Methyl-imidazolidine-2,4-dione.

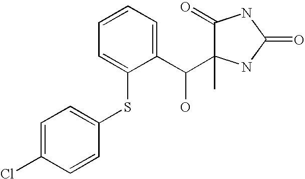

5-{[1-(4-Chloro-phenylH-pyrrol-2-yl]-hydroxy-methyl}-5-methyl-imidazolidine-2,4-dione

Produced by aldol condensation of 1-(4-Chloro-phenyl-1H-pyrrol-2-carbaldehyde and 5-Methyl-imidazolidine-2,4-dione.

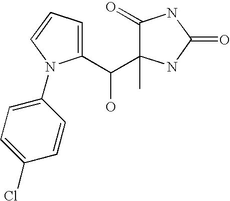

APCI-MS m/z: 302.1 [MH⁺-H₂O]

5-[Hydroxy-(2-pyridin-2-yl-thiophene 2-yl)-methyl]-5-methyl-imidazolidine-2,4-dione

Produced by aldol condensation of 5-pyridin-2-yl-thiophene-2-carbaldehyde and 5-Methyl-imidazolidine-2,4-dione.

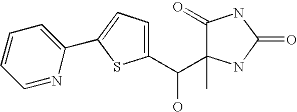

APCI-MS m/z: 304 [MH⁺]

5-[Hydroxy-(5-thiophene-2-H-pyrazol-3-yl)-methyl]-5-methyl-imidazolidine-2,4-dione

Produced by aldol condensation of 5-thiophene-2-yl-2H-pyrazol-3-carbaldehyde and 5-Methyl-imidazolidine-2,4-dione.

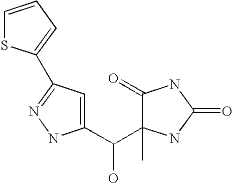

APCI-MS m/z: 293.1 [MH^{+]}

5-{Hydroxy-[5-(4-trifluoromethyl-phenyl H-pyrazol-3-yl]-5-methyl-imidazolidine-2,4-dione

Produced by aldol condensation of 5-(4-trifluoromethyl-phenyl-2H-pyrazol-3-carbaldehyde and 5-Methyl-imidazolidine-2,4-dione.

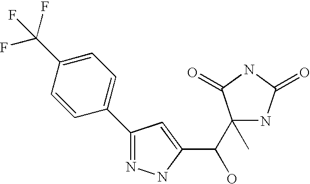

APCI-MS m/z: 355 [MH⁺]

5-(Biphenyl-4-yl-hydroxy-methyl)-5-(4-chloro-benzyl)-imidazolidine-2,4-dione

Produced by aldol condensation of biphenyl-4-carbaldehyde and 5-(4-chloro-benzyl)-imidazolidine-2,4-dione.

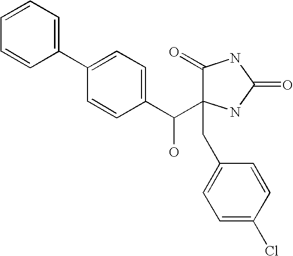

1H NMR (400 MHz, DMSO-d6): δ9.89 (1H, s); 8.29 (1H, s); 7.65 (2H, d, J=7.73 Hz); 7.59 (2H, d, J=8.20 Hz); 7.43 (2H, m); 7.39 (2H, d, J=8.20 Hz); 7.32 (3H, m); 7.20 (2H, d, J=8.39 Hz); 6.13 (1H, d, J=4.01 Hz); 4.85 (1H, d, 4.01 Hz); 3.28 (1H, d, J=13.35 Hz); 3.04 (1H, d, J=13.35).

APCI-MS m/z: 407.2 [MH⁺]

5-Benzylsulfanylmethyl-5-(biphenyl-4-yl-hydroxy-methyl)-imidazolidine-2,4-dione

Produced by aldol condensation of biphenyl-4-carbaldehyde and 5-Benzylsulfanylmethyl-imidazolidine-2,4-dione.

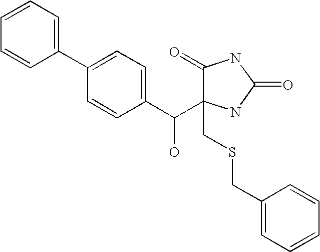

APCI-MS m/z: 419.2 [MH⁺]

5-(Biphenyl-4-yl-hydroxy-methyl)-5-methylsulfanylmethyl-imidazolidine-2,4-dione

Produced by aldol condensation of biphenyl-4-carbaldehyde and 5-methylsulfanylmethyl-imidazolidine-2,4-dione.

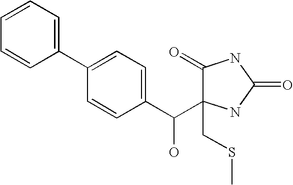

APCI-MS m/z: 343.1 [MH⁺]

5-(Biphenyl-4-yl-hydroxy-methyl)-5-cyclohexylmethyl-imidazolidine-2,4-dione

Produced by aldol condensation of biphenyl-4-carbaldehyde and 5-cyclohexylmethyl-imidazolidine-2,4-dione.

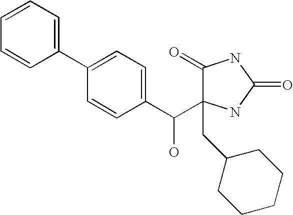

APCI-MS m/z: 379.3 [MH⁺]

5-(Biphenyl-4-yl-hydroxy-methyl)-5phenylethyl-imidazolidine-2,-dione

Produced by aldol condensation of biphenyl-4-carbaldehyde and 5-phenylethyl-imidazolidine-2,4-dione.

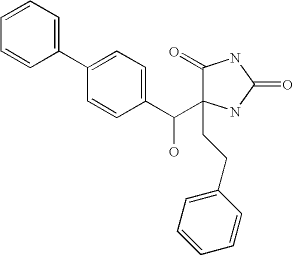

APCI-MS m/z: 387.3 [MH⁺]

5(Biphenyl-4-yl-hydroxy-methyl)-5-2-hydroxy-ethyl)-imidazolidine-2,4-dione

Produced by aldol condensation of biphenyl-4-carbaldehyde and 5-(2-hydroxy-ethyl)-imidazolidine-2,4-dione.

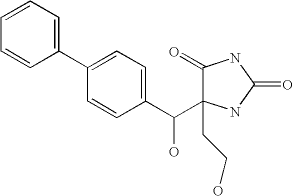

APCI-MS m/z: 309.2 [MH⁺-H₂O]

5-[Hydroxy-(4′-methoxy-biphenyl-4-yl)-methyl]-imidazolidine-2,4-dione

Produced by aldol condensation of 4′-methoxy-biphenyl-4-carbaldehyde and imidazolidine-2,4-dione.

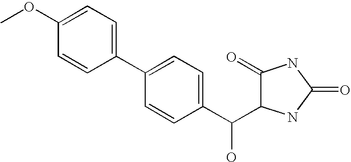

1H NMR (400 MHz, DMSO-d6): δ10.30 (1H, s); 8.06 (1H, s); 7.60 (2H, d, J=8.77 Hz); 7.54 (2H, d, J=8.39 Hz); 7.33 (2H, d, J=8.20 Hz); 7.00 (2H, d, J=8.77 Hz); 5.83 (1H, d, J=3.81 Hz); 4.94 (1H, t, J=3.34); 4.33 (1H, d, J=2.67 Hz); 3.77 (3H, s).

APCI-MS m/z: 295 [MH⁺-H₂O]

5-(Biphenyl-4-yl-hydroxy-methyl)-5-pyridin-4-ylmethyl-imidazolidine-2,4-dione

Produced by aldol condensation of biphenyl-4-carbaldehyde and 5-pyridin-4-ylmethyl-imidazolidine-2,4-dione.

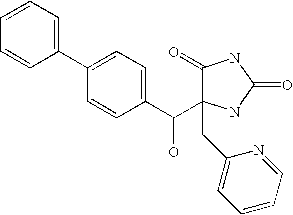

APCI-MS m/z: 374.2 [MH⁺]

5-(Hydroxy-{3-[4-(5-trifluoromethyl-pyridin-2-yl)-piperazin-1-yl]-phenyl}methyl)-5-methyl-imidazolidine-2,4-dione

Produced by aldol condensation of 4-[4-(5-trifluoromethyl-pyridin-2-yl)-piperazin-1-yl]-benzaldehyde and 5-Methyl-imidazolidine-2,4-dione.

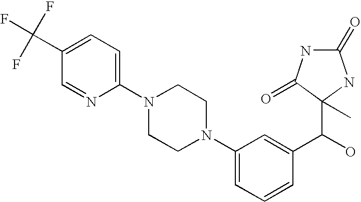

APCI-MS m/z: 450.2 [MH⁺]

5-[(4-}2-[4-(3-Chloro-5-trifluoromethyl-pyridin-2-yl)-piperazin-1-yl]-ethoxy}-phenyl)-hydroxy-methyl]]-5-methyl-imidazolidine-2,4-dione

Prepared from commercially available starting materials.

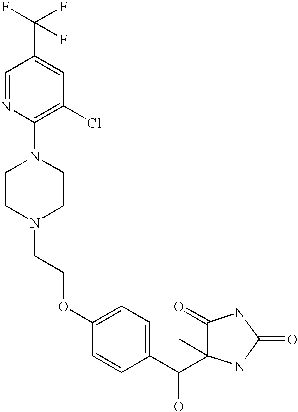

APCI-MS m/z: 528.3 [MH⁺]

### EXAMPLE 9

Compounds were synthesized according to Method D (Suzuki coupling) in Scheme 4 (shown in the description above) from commercially available arylboronic acids and 5-[Hydroxy-(4-iodo-phenyl)-methyl]-5-methyl-imidazolidine-2,4-dione or 5-[Hydroxy-(4-iodo-phenyl)-methyl]-imidazolidine-2,4-dione described below.

5-[Hydroxy-(4-iodo-phenyl)-methyl]-5-methyl-imidazolidine-2,4-dione

4-Iodo-bensaldehyde (9.280 g, 40.0 mmol), 5-methyl-hydantoin (4.564 g, 40.0 mmol) and 45% aques trimethylamine (6.40 ml, 40.0 mmol) was refluxed in ethanol (60 ml) and water (40 ml) for 20 hours under an atmosphere of nitrogen. A white precipitate was formed. After cooling at room temperature for approximately 15 minutes the precipitate was collected by filtration, washed sequentially with ethanol (50%, 50 ml), water (50 ml) and diethyl ether (50 ml). Drying by air suction afforded the title compound 5-[hydroxyl-(4-iodo-phenyl)-methyl]-imidazolidine-2,4-dione (7.968 g, 23.0 mol) in 57.5% yield as white solid in form of the pure racemate.

¹HNMR (300 MHz, DMSO-d₆): δ10.19 (1H, s); 8.08 (1H, s); 7.64 (2H, d, J=8.55 Hz); 7.07 (2H, d, J=8.43 Hz); 5.98 (1H, d, J=4.49 Hz); 4.57 (1H, d, J=4.32 Hz); 1.40 (3H, s).

APCI-MS m/z: 346.9 [MH⁺];

5-[Hydroxy-(4-iodo-phenyl)-methyl]-imidazolidine-2,4-dione

Prepared according to the same protocol used for preparation of 5-[Hydroxy-(4-iodo-phenyl)-methyl]-5-methyl-imidazolidine-2,4-dione described above. ¹HNMR (300 MHz, DMSO-₆): δ10.32 (1H, s); 8.06 (1H, s); 7.66 (2H, d, J=8.14 Hz); 7.10 (2H, d, J=8.27 Hz); 5.91 (1H, d, J=3.90 Hz); 4.87 (1H, t, J=2.70 Hz); 4.34 (1H, d, J=2.48 Hz).

APCI-MS m/z: 333.1 [MH⁺]

4′-[Hydroxy-4-methyl-2,5-dioxo-imidazolidin-4-yl)-methyl]-biphenyl-4-carboxylic acid

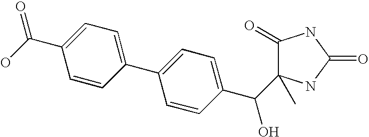

A stirred mixture of 4-Carboxy-phenyl-boronic acid (214 mg, 1.3 mmol), 5-[hydroxy-(4-iodo-phenyl)-methyl]-imidazolidine-2,4-dione (347 mg, 1.0 mmol) and sodium hydrogencarbonate (318 mg, 3.8 mmol) in acetone (5.0 ml) and water (5.0 ml) was deoxygenated by vacuum/nitrogene exchange 3 times. Palladium diacetate (20 mg, yyy mmol) was added and deoxygenating repeated, and then the mixture was stirred at 50° C. for 90 min under an atmosphere of nitrogen.

The solid was allowed to precipitate. The supernatant was partitioned between water (20 ml), ethyl acetate (15 ml) and diethyl ether (15 ml). The water phase was acidified with 1 M HCl (aq, 10 ml) then extracted two times with ethyl acetate (15 ml) and diethyl ether (15 ml). Evaporation of the organic phase afforded 340 mg of the crude product, this was slurred in dioxane (6 ml) and water (6 ml) together with trifluoroacetic acid (100 microl) and filtrated. Preparative HPLC (column, acetonitril/water/trifluoro acetic acid) afforded the title compound 4′-[Hydroxy-(4-methyl-2,5-dioxo-imidazolidin-4-yl)-methyl]-biphenyl-4-carboxylic acid (114 mg, 0.33 mmol) as a white solid in 33.5% yield.

¹HNMR (400 MHz, DMSO-d₆): δ10.20 (1H, s); 8.13 (1H, s); 8.00 (2H, d, J=8.33 Hz); 7.79 (2H, d, J=8.49 Hz); 7.67 (2H, d,J=8.39 Hz); 7.40 (2H, d, J=8.48 Hz); 5.97 (1H, bs); 4.68 (1H, s); 1.44 (3H, s).

APCI-MS m/z: 341 [MH⁺]

The following compounds where prepared by the same protocol used for preparation of 4′-[Hydroxy-(4-methyl-2,5-dioxo-imidazolidin-4-yl)-methyl]-biphenyl-4-carboxylic acid described above.

5-[Hydroxy-(4′-methylsulfanyl-biphenyl-4-yl)-methyl]-5-methyl-imidazolidine-2,4-dione

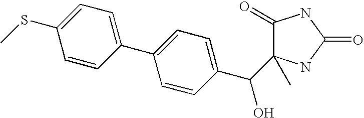

¹HNMR (300 MHz, DMSO-d₆): δ10.18 (1H, s); 8.10 (1H, s); 7.62 (2H, d, J=8.61 Hz); 7.57 (2H, d, J=8.42 Hz); 7.35 (2H, d, J=5.73 Hz); 7.32 (2H, d, J=6.30 Hz); 5.91 (1H, d, J=4.32 Hz); 4.65 (1H, d, J=4.31 Hz); 2.50 (3H, s); 1.43 (3H, s).

APCI-MS m/z: 343.0 [MH⁺]

5-[Hydroxy-(4-naphtalen-2-yl phenyl)-methyl]-5-methyl-imidazolidine-2,4-dione

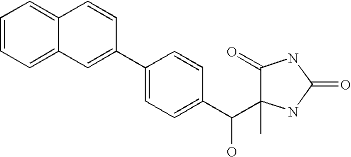

APCI-MS m/z: 347.1 [MH⁺]

5-[Hydroxy-[1,1′;4,1″]terpenyl-4″-yl-methyl)-5-methyl-imidazolidine-2,4-dione

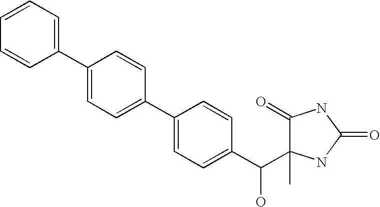

APCI-MS m/z: 373.1 [MH⁺]

5-[(3′-Benzyloxy-biphenyl-4-yl)-hydroxy-methyl]-5-methyl-imidazolidine-2,4-dione

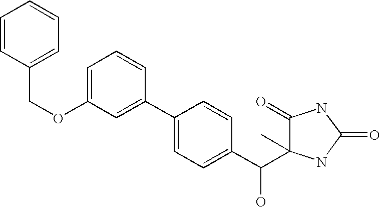

APCI-MS m/z: 403.1 [MH⁺].

5-[(4-Benzo[1,3]dioxol-5-yl-phenyl)-hydroxy-methyl]-imidazolidine-2,4-dione

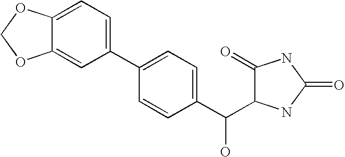

1H NMR (400 MHz, DMSO-d6): δ10.31 (1H, s); 8.04 (1H, s); 7.53 (2H, d, J=8.39 Hz); 7.33 (2H, d, J=8.20 Hz); 7.24 (1H, s); 7.14 (1H, d, J=8.11 Hz); 6.97 (1H, d, J=8.01 Hz); 6.03 (2H, d, J=6.87 Hz); 5.84 (1H, d, J=3.62 Hz); 4.92 (1H, s); 4.35 (1H, s).

APCI-MS m/z: 309 [MH⁺-H₂O]

5-[Hydroxy-(3′-nitro-biphenyl-4-yl)-methyl]-5-methyl-imidazolidine-2,4-dione

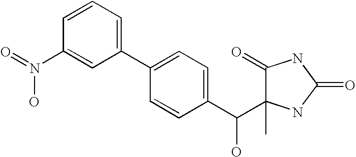

1H NMR (400 MHz, DMSO-d6): δ10.18 (1H, s); 8.41 (1H, t, J=8.41 Hz); 8.20 (1H, m); 8.15 (1H, m); 8.12 (1H, s); 7.73 (3H, m); 7.41 (2H, d, J=8.20); 5.97 (1H, d, J=4.39 Hz); 4.68 (1H, d, 4.58 Hz); 1.43 (3H, s).

APCI-MS m/z: 342.1 [MH⁺]

### EXAMPLE 10

Compounds were synthesized according to Method E (Amide coupling) in Scheme 4 (shown in the description above). The compounds were prepared by the general method described below; All amines used in the coupling are commercially available.

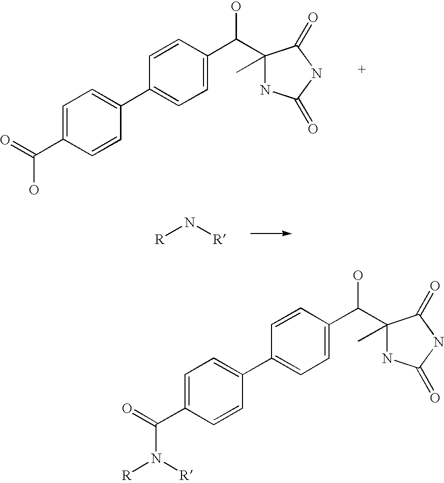

To a 0.3M solution of 4′-[hydroxy-(4-methyl-2,5-dioxo-imidazolidin-4-yl)-methyl]-biphenyl-4-carboxylic acid in 1-methyl-2-pyrrolidinone (50 μL) was 1-ethyl-3(3-dimethylaminopropyl)carbdiimide hydrochloride (1.3 eq ,45 μL 0.5M in 1-methyl-2-pyrrolidinone), 1-hydroxybenzotriazole (1.7 eq, 51 μL 0.5M in 1-methyl-2-pyrrolidinone), N,N-diisopropylethylamine (1 eq , 20 μL 1M in 1-methyl-2-pyrrolidinone) and the corresponding amine ( 2 eq, 100 μL 0.3M in 1-methyl-2-pyrrolidinone) added. The reaction mixture was stirred over night at room temperature. Purification was made by preparative HPLC-C₁₈.

4′-[Hydroxy-(4-methyl-2,5-dioxo-imidazolidin-4-yl)-methyl]-biphenyl-4-carboxylic acid (2-hydroxy-ethyl)-methyl-amide

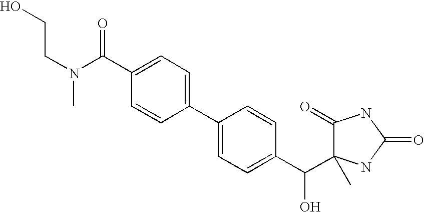

APCI-MS m/z: 398.1 [MH⁺]

5-{Hydroxy-[4′-(morpholine-4-carbonyl)-biphenyl-4-yl]-methyl}-5-methyl-imidazolidine-2,4-dione

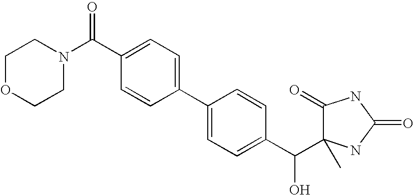

APCI-MS m/z: 410.1 [MH⁺]

4′-[Hydroxy-4-methyl-2,5-dioxo-imidazolidin-4-yl)-methyl]-biphenyl-4-carboxylic acid methyl-(1-methyl-pyrrolidin-3-yl)-amide

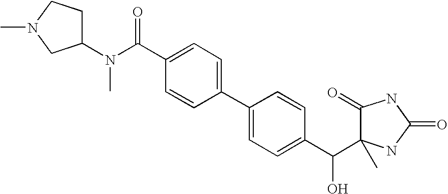

APCI-MS m/z: 437.1 [MH⁺]

4′-[Hydroxy-(4-methyl-2,5-dioxo-imidazolidin-4-yl)-methyl]-biphenyl-4-carboxylic acid (2-morpholinyl-ethyl)-amide

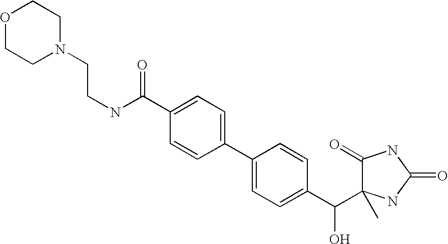

APCI-MS m/z: 453.1 [MH⁺]

4′-[Hydroxy-(4-methyl-2,5-dioxo-imidazolidin-4-yl)-methyl]-biphenyl-4-carboxylic acid (2-methoxy-ethyl)-amide

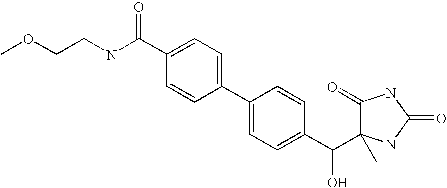

APCI-MS m/z: 398.1 [MH⁺]

5-{(]hydroxy-[4′-(pyrrolidine-1-carbonyl)-biphenyl-4-yl]-methyl}-5-methyl-imidazolidine-2,4-dione

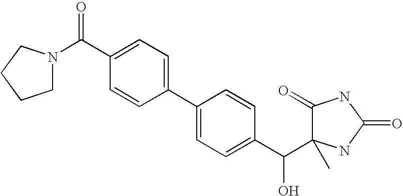

APCI-MS m/z: 394.1 [MH⁺]

4′-[Hydroxy-(4-methyl-2,5-dioxo-imidazolidin-4-yl)-methyl]-biphenyl-4-carboxylic acid (2-cyano-ethyl)-methyl-amide

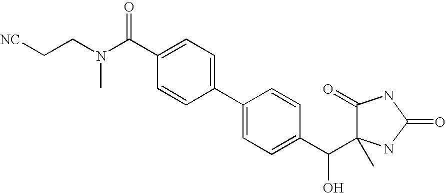

APCI-MS m/z: 407.1 [MH⁺]

4′-[Hydroxy-(4-methyl-2,5-dioxo-imidazolidin-4-yl)-methyl]-biphenyl-4-carboxylic acid methyl-phenethyl-amide

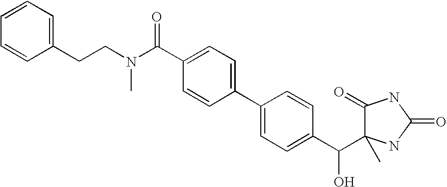

APCI-MS m/z: 458.1 [MH⁺]

4′-[Hydroxy-(4-methyl-2,5-dioxo-imidazolidin-4-yl)-methyl]-biphenyl-4-carboxylic acid (4-cyano-cyclohexyl)-methyl-amide

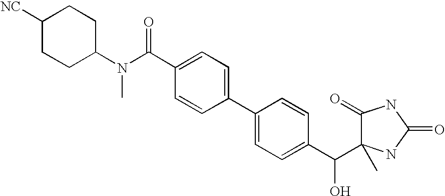

APCI-MS m/z: 461.1 [MH⁺]

5-{Hydroxy-[4′-(4-hydroxymethyl-piperidine-1-carbonyl)-biphenyl-4-yl]-methyl-1}-5-methyl-imidazolidine-2,4-dione

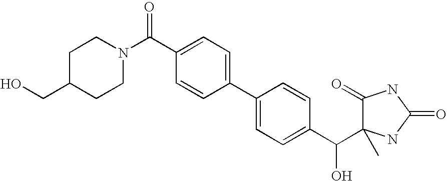

APCI-MS m/z: 438.1 [MH⁺]

4-[Hydroxy-(4-methyl-2,5-dioxo-imidazolidin-4-yl)-methyl]-biphenyl-4-carboxylic acid [3-(2-oxo-pyrrolidin-1-yl)-propyl]-amide

APCI-MS m/z: 465.1 [MH⁺]

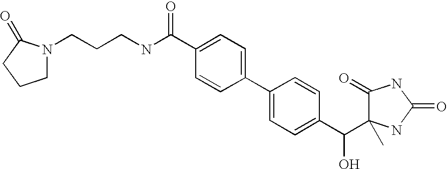

4′-[Hydroxy-(4-methyl-2,5-dioxo-imidazolidin-4-yl)-methyl]-biphenyl-4-carboxylc acid cyclopentylamide

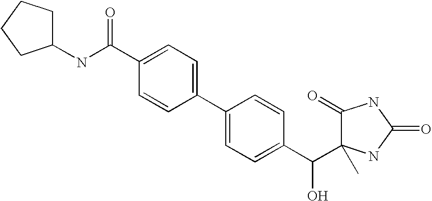

APCI-MS m/z: 408.1 [MH⁺]

4′-[Hydroxy-(4-methyl-2,5-dioxo-imidazolidin-4-yl)-methyl]-biphenyl-4-carboxylc acid (1-phenyl-ethyl)-amide

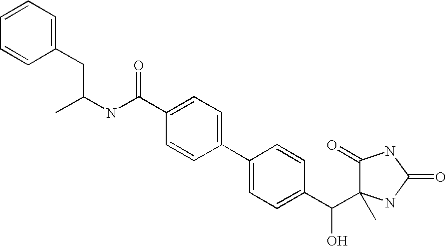

APCI-MS m/z: 444.1 [MH⁺]

4′-[Hydroxy-(4-methyl-2,5-dioxo-imidazolidin-4-yl)-methyl]-biphenyl-4-carboxylic acid (pyridin-4-ylmethyl)-amide

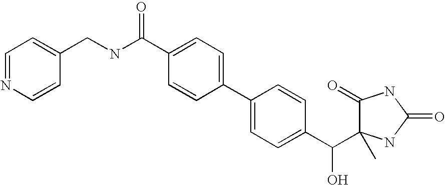

APCI-MS m/z: 431.1 [MH⁺]

4′-[Hydroxy-(4-methyl-2,5-dioxo-imidazolidin-4-yl)-methyl]-biphenyl-4-carboxylic acid benzylamine

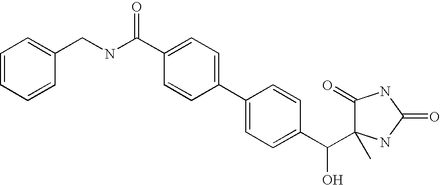

APCI-MS m/z: 430.1 [MH⁺]

4′-[Hydroxy-(4-methyl-2,5-dioxo-imidazolidin-4-yl)-methyl]-biphenyl-4-carboxylic acid cyclopropylamide

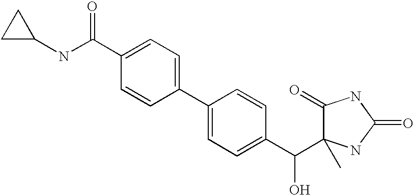

APCI-MS m/z4380.1 [MH⁺]

4′-[Hydroxy-(4-methyl-2,5-dioxo-imidazolidin-4-yl)-methyl]-biphenyl-4-carboxylic acid 4-methoxy-benzylamide

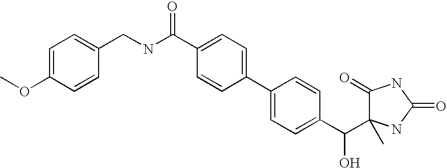

APCI-MS m/z: 460.1 [MH⁺]

4′-[Hydroxy-(4-methyl-2,5-dioxo-imidazolidin-4-yl)-methyl]-biphenyl-4-carboxylic acid (3-imidazol-1-yl-propylamide

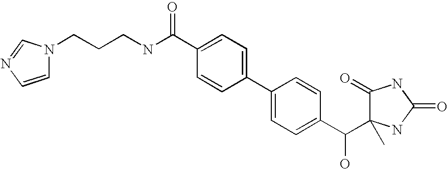

APCI-MS m/z: 448.1 [MH⁺]

N-{4-[Hydroxy-(4-methyl-2,5-dioxo-imidazolidin-4-yl)-methyl]-phenyl}-benzamide

5-[Hydroxy-(4-nitro-phenyl)-methyl]-5-methyl-imidazolidine-2,4-dione was synthesized according to method C by the protocol described in Example 1(APCI-MS m/z: 268.8 [MH⁺]). The corresponding amine 5-[(4-Amino-phenyl)-hydroxy-methyl]-5-methyl-imidazolidine-2,4-dione was afforded by Pd(0) catalysed hydrogenation in Ethanol (

APCI-MS m/z: 218.0 [MH⁺] (-H20)). 5-[(4-Amino-phenyl)-hydroxy-methyl]-5-methyl-imidazolidine-2,4-dione was finaly coupled with benzoic acid according to the protocol above (Method E) to afford the title compound.

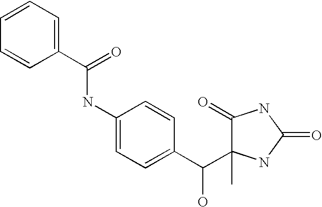

APCI-MS m/z: 240.0 [MH⁺]

### EXAMPLE 11

Enantiomeres where isolated by the method described for the resolution of 4′-(hydroxy-(4-methyl-2,5-dioxoimidazolidin-4-yl)-methyl)biphenyl-4-carbonitrile below.

4′-(hydroxy-(4-methyl-2,5-dioxoimidazolidin-4-yl)-methyl)biphenyl-4-carbonitrile

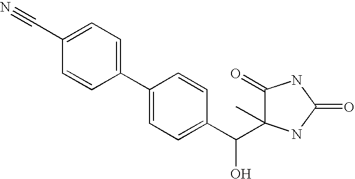

Chromatographic Resolution:

0.10 g of diastereomerically pure 4′-(hydroxy-(4-methyl-2,5-dioxoimidazolidin-4-yl)-methyl)biphenyl-4-carbonitrile was dissolved in 76 mL absolute ethanol/iso-hexane (75:25) and filtered through a 0.45 μm nylon filter. Volumes of 5.0 mL were injected repeatedly on a chiral column (Chiralpak AD-H (2 cm ID×25 cm L)) connected to a UV-detector (254 nm) and fraction collector. Separation was performed with absolute ethanol/is-hexane (75:25) at 8.0 mol/min flow and the pure enantiomers eluted after approximately 15 and 21 minutes, respectively. Fractions containing the same enantiomer were combined, concentrated and assessed for optical purity by chiral chromatography (see below).

Enantiomer A (“Early” Fractions)

Yield: 0.047 g white solid

Chiral chromatography (Chiralpak AD-H (0.45 cm I.D×25 cm L) at 0.43 mL/min absolute ethanol/iso-hexane (75:25))

Retention time: 11.4 minutes

Optical purity: 99.9% e.e (no enantiomer B present)

¹H NMR (CD₃OD) δ1.60 (s, 3H), 4.84 (m obscured by water singlett, 1H), 7.50 (d, 2H, J=8 Hz), 7.62 (d, 2H; J=8 Hz) and 7.79 (m, 4H) ppm.

Enantiomer B (“Late” Fractions)

Yield: 0.040 g white solid

Chiral chromatography (Chiralpak AD-H (0.45 cm I.D×25 cm L) at 0.43 ml/min absolute ethanol/iso-hexane (75:25))

Retention time: 18.0 minutes

Optical purity: 99.0% e.e (0.50% of enantiomer A present)

¹H NMR (CD₃OD) δ1.60 (s, 3H), 4.84 (m obscured by water singlett, 1H), 7.50 (d, 2H, J=8 Hz), 7.62 (d, 2H; J=8 Hz) and 7.79 (m, 4H) ppm.

N-(4′-(hydroxy-(4-methyl-2,5-dioxoimidazolidin-4-yl)-methyl)biphenyl-3-yl)acetamide

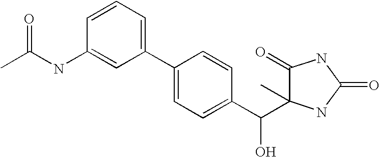

Chromatographic Resolution:

0.040 g of diastereomerically pure N-4′-(hydroxy-(4-methyl-2,5-dioxoimidazolidin-4-yl)-methyl)biphenyl-3-yl)acetamide was dissolved in 224 mL absolute ethanol/iso-hexane (71:29) and separated as discribed above with absolute ethanol iso-hexane (50:50) at 6.0 mL/min as eluant.

Enantiomer A (“Early” Fractions)

Yield: 0.019 g white solid

Chiral chromatography (Chiralpak AD-H (0.45 cm I.D×25 cm L) at 0.43 mL/min absolute ethanol iso-hexane (50:50))

Retention time: 10.4 minutes

Optical purity: 99.9% e.e (no enantiomer B present)

¹H NMR (CD₃OD) δ1.60 (s, 3H), 2.14 (s, 3H), 4.82 (m obscured by water singlett, 1H), 7.33 (m, 1H), 7.36 (t, 1H, J=8 Hz), 7.44 (d, 2H, J=8 Hz), 7.50 (m, 1H), 7.54 (d, 2H, J=8 Hz) and 7.82 (m, 1H) ppm.

Enantiomer B (“Late” Fractions)

Yield: 0.018 g white solid

Chiral chromatography (Chiralpak AD-H (0.45 cm I.D×25 cm L) at 0.43 mL/min absolute ethanol/iso-hexane (50:50))

Retention time: 14.8 minutes

Optical purity: 99.6% e.e (0.20% of enantiomer A present)

¹H NMR (CD₃OD) δ1.60 (s, 3H), 2.14 (s, 3H), 4.82 (m obscured by water singlett, 1H), 7.33 (m, 1H), 7.36 (t, 1H, J=8 Hz), 7.44 (d, 2H, J=8 Hz), 7.50 (m, 1H), 7.54 (d, 2H, J=8 Hz) and 7.82 (m, 1H) ppm. 5-(Biphenyl-4-yl-hydroxy-methyl)-imidazolidine-2,4-dione.

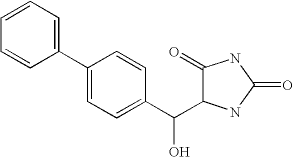

Chromatographic Resolution:

Separation was made on a Gilson HPLC system (column: CHIRALPAK AD, 2.0×25 cm. Solvent: isoHexane/EtOH=25/75. Flow=6.0 mL/min. WV=254 nm. Inj volume=3.0 mL). 24 mg of the racemic material was dissolved in 24 mL of isoHexane/EtOH=25/75. The two enantiomers with Rt=17.72 min and 20.47 min was collected and solvent was removed by evaporation. Analysed for enantiomeric purity using the following Gilson HPLC system (column: CHIRALPAK AD, 0.46×25 cm. Solvent: isoHexane/EtOH=25/75. Flow=0.5 mL/min. UV=254 nm).Faster enantiomer: 9 mg, Rt=10.12 min, ee=99.9%. Slower enantiomer: 7 mg, Rt=11.78 min, ee=99.2%.

### EXAMPLE 12

The following compounds where prepared by a method analogous to that described in Example 1.

5-[(9 H-Fluoren-2-yl)-hydroxy-methyl]-imidazolidine-2,4-dione

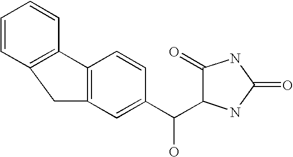

APCI-MS m/z: 277 [MH+-H2O]

(3-{4-[(4′-Fluoro-biphenyl-4-yl)-hydroxy-methyl]-2,5-dioxo-imidazolidin-4-yl}-propyl)carbamic acid benzyl ester

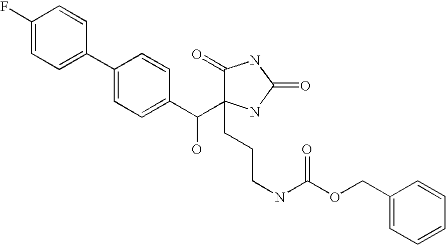

1H NMR (400 MHz, DMSO-d6): δ10.20 (1H, s); 8.53 (1H,d, J=4.01 Hz); 8.01 (1H, s); 7.69 (2H, m); 7.56 (2H, d, J=8.39 Hz), 7.30 (9H, m), 5.90 (1H, d, J=4.20 Hz), 4.99 (2H, s) 4.64 (1H, d, J=4.20 Hz); 2.98(2H, m), 1.97 (1H, m), 1.72 (1H, m), 1.42 (1H, m), 1.22 (1H, m).

APCI-MS m/z: 492.2 [MH⁺]

5-(3-Amino-propyl)-5-[(4′-fluoro-biphenyl-4-yl)-hydroxy-methyl]-imidazolidine-2,4-dione

Prepared from above (3-{4-[(4′-Fluoro-biphenyl-4-yl)-hydroxy-methyl]-2,5-dioxo-imidazolidin-4-yl}-propyl)-carbamic acid benzyl ester by a standard method known for those skilled in the art.

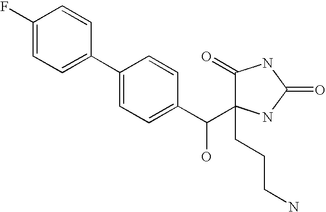

APCI-MS m/z: 358.1 [MH⁺]

5-[Hydroxy-4′methoxy-biphenyl-4-yl)-methyl]-5-methylsulfanylmethyl-imidazolidine-2,4-dione

Prepared from 4′-methoxy-biphenylcarbaldehyde (Table 3, Method B) and 5-methylsulfanylmethyl-imidazolidine-2,4.dione (Table 2, Method A) according to Method C, Example 1.

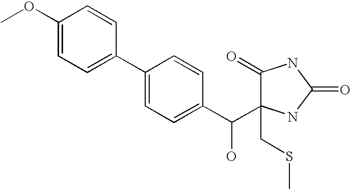

1H NMR (400 MHz, DMSO-d6): δ10.25 (1H, s); 8.16 (1H, s); 7.59 (2H, d, J=8.77 Hz,), 7.53(2H, d, J=8.20 Hz); 7.31 (2H, d, J=8.20 Hz); 6.99 (2H, d, J=8.77 Hz); 5.98 (1H, d, J=4.20 Hz); 4.71 (1H, d, J=4.01 Hz); 3.77 (3H, s); 3.16 (1H, d, J=14.31 Hz9, 2.92(1H, d, J=14.3 1 Hz), 2.11 (3H, s).

APCI-MS m/z: 373.1 [MH⁺]

5-[Hydroxy-(4′-methoxy-biphenyl-4-yl)-methyl]-5-pyridin-2-ylmethyl-imidazolidine-2,4-dione

Prepared from-4′-methoxy-biphenylcarbaldehyde (Table 3, Method B) and commercially available 5-pyridin-2-ylmethyl-imidazolidine-2,4.dione according to Method C, Example 1.

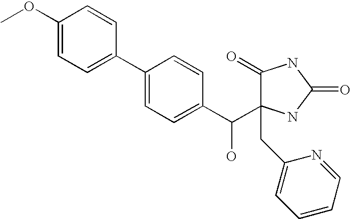

1H NMR (400 MHz, DMSO-d6): δ10.00 (1H, s); 8.53 (1H,d, J=4.01 Hz); 8.13 (1H, s,); 7.91 (1H, s); 7.58 (2H, m); 7.53 (2H, m); 7.38 (4H, m), 7.00 (2H, m), 6.11 (1H, s) 4.81 (1H, s); 3.48(2H, m).

APCI-MS m/z: 404.3 [MH⁺]

5-[Hydroxy-(4-pyrazin-2-yl-phenyl)-methyl]-5-methyl-imidazolidine-2,4-dione

Prepared from commercially available 4-pyrazin-2-yl-benzaldehyde and 5-methyl-hydantoin according to Method C, Example 1.

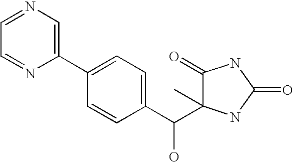

APCI-MS m/z: 299 [MH⁺].

5-{3-[4-(5-Chloro-pyridin-2-yloxy)-phenyl]-1-hydroxy-propyl}-5-methyl-imidazolidine-2,4-dione

3-[4-(5-Chloro-pyridin-2-yloxy)-phenyl]-propanol-1-ol

3-(4-Hydroxyphenyl)-propanol (768.5, 5.05 mmol), 2,5-dichloro-pyridine (934.8 mg, 6.32 mmol), cesium carbonate (2.48 g, 7.60 mmol) mixed in N-methyl-pyrollidone (10 ml) was stirred and heated (100° C.) for 20 hours. The flask was cooled and the content was partitioned between ethyl acetate (100 ml), di-tertbutylether (100 ml) and water (300 ml). The organic phase was washed with water (3×30 ml). Evaporation afforded the crude title compound (1.502 g, 5.70 mmol) as a yellow oil in 113% yield. Pure according to TLC analysis.

APCI-MS m/z: 264 [MH⁺]

3-[4-(5-Chloro-pyridin-2-yloxy)-phenyl]-propionaldehyde

3-[4-(5-Chloro-pyridin-2-yloxy)-phenyl]-propanol-1-ol (267 mg, 1.0 mmol) and pyridinium chloro chromate (302 mg, 1.4 mmol) was stirred in dichloromethan (20 ml, molecular sieve dried) for 2 hours. Flash chromatography (SiO2, dichloromethan/methanol: gradient to 100/5) afforded the title compound (169 mg, 0.65 mmol) as a oil in 65% yield.

APCI-MS m/z: 262 [MH⁺]

5-{3-[4-(5-Chloro-pyridin-2-yloxy)-phenyl]-1-hydroxy-propyl}-5-methyl-imidazolidine-2,4-dione

3-[4-(5-Chloro-pyridin-2-yloxy)-phenyl]-propionaldehyde and commercially available 5-methyl-hydantoin was utilized for synthesis of the title compound according to Method C, Example 1.

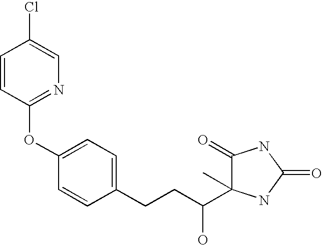

APCI-MS m/z: 376.0 [MH⁺]

5-{[4-(5-Chloro-pyridin-2-yloxy)-phenyl]-hydroxy-methyl}-5-methyl-imidazolidine-2,4-dione

4-(5-Chloro-pyridin-2-yloxy)-benzaldehyde

4-Hydroxy-benzaldehyde (620.9 mg, 5.08 mmol), cesiumcarbonate (2.6 g, 7.98 mmol) and 2,5-dikloropyridine (947 mg, 6.40 mmol) mixed in N-methyl-pyrollidone (10 ml) was stirred and heated (75° C.) for 16 hours. LCMS analysis indicated formation of product in a minor amount Further reaction at elevated temperature (150° C.) for additional six hours produced increased formation of product The flask was cooled and the content was partitioned between ethyl acetate (100 ml), ether (100 ml) and water (200 ml). The organic phase was washed with water (3×30 ml). Evaporation and flash chromatography (SiO₂, dichloromethan/methanol: gradient to 100/4) afforded 4-(5-chloro-pyridin-2-yloxy)-benzaldehyde (181 mg, 0.77 mmol) in 15.2% yield.

1H NMR (460 MHz, DMSO-d6): δ9.98 (1H, s);8.27 (1H, d);8.04 (1H, dd);7.97 (2H, d);7.35 (2h, d);7.23 (1H, d).

APCI-MS m/z: 234 [MH⁺]

5-{[4-(5-Chloro-pyridin-2-yloxy)-phenyl]-hydroxy-methyl}-5-methyl-imidazolidine-2,4-dione

4-(5-Chloro-pyridin-2-yloxy)-benzaldehyde and commercially available 5-methyl-hydantoin was utilized for synthesis of the title compound according to Method C, Example 1.

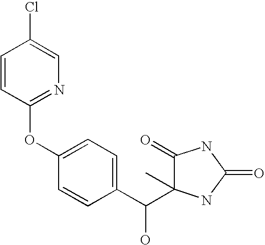

APCI-MS m/z: 348 [MH⁺]

### EXAMPLE 13

5-[(3′-Amino-biphenyl-4-yl)-hydroxy-methyl]-5-methyl-imidazolidine-2,4-dione

Prepared from 5-[Hydroxy-(3′-nitro-biphenyl-4-yl)-methyl]-5-methyl-imidazolidine-2,4-dione described in Example 8 by by a standard synthetic method well-known for those skilled in the art (Pd (0) catalysed hydrogenation in ethanol).

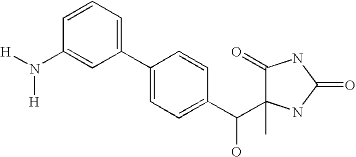

APCI-MS m/z: 312.1 [MH⁺]

### EXAMPLE 14

The following compounds where prepared according to the protocol used for synthesis of N-{4′-[hydroxy-(4-methyl-2,5-dioxo-imidazolin-4-yl)-methyl]-biphenyl-3-yl}-methansulfonamide described below.

N-{4′-[hydroxy-(4-methyl-2,5-dioxo-imidazolin-4-yl)-methyl]-biphenyl-3-yl)-methansulfonamide

Methanesulfonyl chloride (10 ul, 0.165 mmol) was added dropwise to a solution of 5-[(3′-Amino-biphenyl-4-yl)-hydroxy-methyl]-5-methyl-imidazolidine-2,4-dione (41 mg, 0.132 mmol) in pyridine (1 ml). The resulting mixture was stirred for 6 hours at ambient temperature. Water (15 ml) was added and the aqueous mixture was extracted with EtOAc (3×10 ml). The combined EtOAc extracts were dried (MgSO₄) and concentrated under reduced pressure to afford the crude product. Preparative HPLC on a Chromasil C18 column with acetonitrile/water (0.1% trifluoroacetic acid), afforded the 40 mg (80% yield) of the title compound N-{4′-[hydroxy-(4-methyl-2,5-dioxo-imidazolin-4-yl)-methyl]-biphenyl-3-yl}-methansulfonamide.

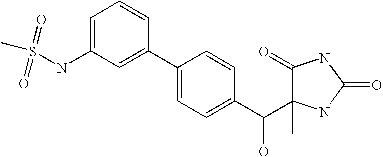

1H NMR (400 MHz, DMSO-d6): δ10.17 (1H, s); 9.79 (1H,s); 8;10 (1H, s,); 7.57 (2H, d, J=8.39 Hz); 7.40 (5H, m); 7.19 (1H, m); 7.25 (2H, d, J=8.39 Hz); 7.20 (1H, m); 5.92 (1H, m); 4.65 (1H, s); 3.01 (3H, s); 1.42 (3H, s,).

APCI-MS m/z: 390.1 [MH⁺]

N-{4′-[hydroxy-(4-methyl-2,5-dioxo-imidazolin-4-yl)-methyl]-biphenyl-3-yl}-propionate

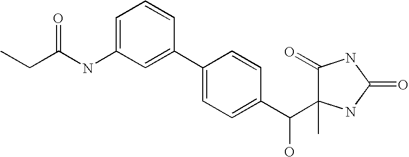

1H NMR (400 MHz, DMSO-6): δ10.17 (1H, s); 9.90 (1H,s); 8.09 (1H, s,); 7.90 (1H, s); 7.51 (3H, m); 7.32 (4H, m); 5.92 (1H, d, J=4.39 Hz); 4.65 (1H, d, J=4.39Hz); 2.32 (1H, q, J=7.44 Hz); 1.42(3H, s); 1.08 (3H, t, J=7.53 Hz).

APCI-MS m/z: 368.1 [MH⁺].

N-{4′-[hydroxy-(4-methyl-2,5-dioxo-imidazolin-4-yl)-methyl]-biphenyl-3-yl}-isobutyramide

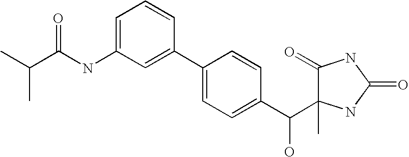

1H NMR (400 MHz, DMSO-d6): δ10.15 (1H, s); 9.87 (1H,s); 8.09 (1H, s,); 7.92 (1H, s); 7.52 (3H, m); 7.33 (4H, m); 5.92 (1H, d, J=4.39 Hz); 4.65 (1H, d, J=4.39 Hz); 2.59 (1H, m); 1.42(3H, s); 1.10 (6H, d, J=6.87 Hz).

APCI-MS m/z: 382.1 [MH⁺].

N-(4′-[hydroxy-(4-methyl-2,5-dioxo-imidazolin-4-yl)-methyl]-biphenyl-3-yl}-2,2-dimethylpropionamide

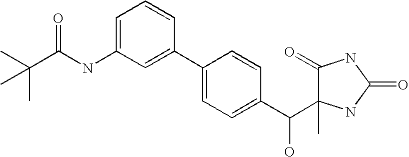

1H NMR (400 MHz, DMSO-d6): δ10.15 (1H, s); 9.23 (1H,s); 8.09 (1H, s,); 7.93 (1H,s); 7.58 (3H, m); 7.33 (4H, m); 5.91 ((1H, d, J=4.39 Hz); 4.65 (1H, d, J=4.39 Hz); 1.42 (3H, s); 1.22 (9H, s).

APCI-MS m/z: 396.2[MH⁺]

### EXAMPLE 15

5-[(4′-Chlorobiphenyl-4-yl)methoxymethyl]-5-methylimidazolidine-2,4-dione

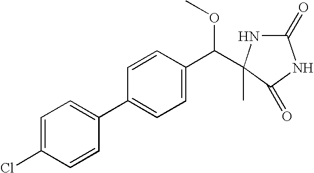

4-Chloro-4′-(2-nitropropenyl)biphenyl

4-(4-Chlorophenyl)benzaldehyde (0.66 g, 3.0 mmoles), nitroethane (2 mL), ammonium carbonate (3.5 g) and glacial acetic acid (17 mL) was stirred under nitrogen at 82° C. for 20 hours. Volatiles were evaporated, the wellow residue was taken up in ether and washed once with water. The aqueous phase was separated and washed once with ether. The combined organic phases were washed with water and brine, dried over anhydrous sodium sulfate, filtered and concentrated with silica (3 g) by rotary evaporation. The dry residue was applied on a silica column. Elution with ethyl acetate/n-heptane (1:20) through (1:8) gave 0.50 g (61% yield) of the title compound as wellow crystalls. Mp. 113.8-114.3° C. (uncorrected).

FT-IR (ATR) v 1647 (w), 1504 (str), 1484 (str), 1320 (v str), 812 (str) cm⁻¹. ¹H NMR (300 MHz, CDCl₃) δ2.50 (d, 3H, J=1 Hz), 7.44 (d, 2H, J=9 Hz), 7.52 (d, 2H, J=9 Hz), 7.55 (d, 2H, J=9 Hz), 7.65 (d, 2H, J=9 Hz) and 8.12 (br s, 1H) ppm. ¹³C NMR (100 MHz, CDCl₃) δ14.2, 127.2, 128.2, 129.1, 130.5, 131.5, 132.9, 134.1, 138.1, 141.3 and 147.6 ppm.

4-Chloro-4-(1-methoxy-2-nitropropyl)biphenyl

A mixture of 4-chloro-4′-(2-nitropropenyl)biphenyl (0.39 g, 1.3 mmoles), sodium is methoxide (4.0 mmoles; freshly prepared from 0.091 g of sodium and dry methanol) and anhydrous 1,2-dimethoxyethane (3.0 mL) was stirred under nitrogen at 22° C. for three hours, acidified with 10% acetic acid in methanol (4 mL), concentrated to dryness by rotary evaporation and then taken up in ethyl acetate and water. The aqueous phase was separated and washed once with ethyl acetate. The combined organic phases were washed with brine, dried over anhydrous sodium sulfate, filtered and concentrated with silica (3 g) by rotary evaporation. The dry residue was applied on a silica column. Elution with dichloromethane/n-heptane (1:3) through (1:1) gave 0.40 g (95% yield) of the title compound as a white solid.

FT-IR (ATR) v 1552 (v str), 1485 (str), 1092 (str), 814 (str) cm⁻¹. ¹H NMR (400 MHz, CDCl₃) δ1.30 (d, 1.3 H, J=7 Hz) 1.56 (d, 1.7 H, J=7 Hz), 3.22 (s, 1.2 H), 3.32 (s, 1.8 H), 4.56 (d, 1.2 H,J=10 Hz), 4.63 (m_{c}, 1.8 H), 4.76 (m^{c}, 1.2 H), 4.88 (d, 1.8 H, J=5 Hz) and 7.38-7.62 (ms, 8 H) ppm. ¹³C NMR (100 MHz, CDCl₃) δ13.0, 16.3, 57.0, 57.7, 83.5, 84.8, 86.9, 87.5, 127.3, 127.5, 128.3, 129.0, 129.1, 132.7, 133.7, 133.9, 135.1, 135.9, 138.7, 138.8, 140.4, 140.9 ppm (diastereomeric signals).

1-(4′-Chlorobiphenyl-4-yl)-1-methoxypropan-2-one

A mixture of 4-chloro-4′-(1-methoxy-2-nitropropyl)biphenyl (0.123 g, 0.40 mmoles), dry dichloromethane (2.8 mL) and finely ground 3A molecular seeves (0.040 g) under argon was cooled on an ice bath. Tetrapropylammonium perruthenate (0.170 g, 0.48 mmoles) was added in a portionwise manner to the cold, stirred mixture. When the addition was completed, the ice bath was removed and the mixture was stirred at 22° C. for 4.0 hours. Diethyl ether (30 mL) was added and the resulting dark suspension was filtered through Celite. The clear filtrate was concentrated with silica (4 g) by rotary evaporation. The dry residue was applied on a silica column. Elution with dichloro-methane/n-heptane (1:2) through (2:1) gave 0.052 g (47% yield) of the title compound as a white solid. FT-IR (ATR) v 1716 (v str), 1485 (str), 1093 cm⁻¹ (v str).

¹H NMR (300 MHz, CDCl₃) δ2.16 (s, 3 H) 3.42 (s, 3 H), 4.69 (s, 1 H), 7.40 (d, 2 H, J=9 Hz), 7.46 (d, 2 H, J=8 Hz), 7.51 (d, 2 H, J=9 Hz) and 7.56 (d, 2 H, J=8 Hz) ppm. ¹³C NMR (100 MHz, CDCl₃) δ25.1, 57.3, 89.1, 127.2, 127.4, 128.2, 128.8, 133.5, 135.1, 138.8, 140.1 and 206.4 ppm

5-[(4′-Chlorobiphenyl-4-yl)methoxymethyl]-5-methylimidazolidine-2,4-dione

1-(4′-Chlorobiphenyl-4-yl)-1-methoxypropan-2-one (0.051 g, 0.19 mmoles), ammonium carbonate (0.089 g, 0.93 mmoles), potassium cyanide (0.025 g, 0.37 mmoles; CAUTION!) and 50% ethanol in water (1.4 mL) were stirred in a sealed vial (4.5 mL) at 87° C. (oil bath temperature) for 19 hours. The solvent was evaporated, water was added to make a volume of approx. 20 mL, pH was adjusted to 3 with glacial acetic and the crude product was taken up in ethyl acetate (50 mL). The organic phase was washed once with brine, dried over anhydrous sodium sulfate, filtered and concentrated by rotary evaporation to afford 0.065 g (100% yield) of the title compound as a white solid. ¹H NMR (400 MHz, DMSO-d₆) δ1.06 (s, 2 H), 1.43 (s, 1 H), 3.07 (s, 2 H), 3.17 (s, 1 H), 4.33 (s, 0.7 H), 4.34 (s, 0.3 H), 7.30-7.75 (m's, 8.7 H), 8.24 (br s, 0.3 H), 10.26 (br s, 0.3 H) and 10.56 (br s, 0.7 H) ppm. ¹³C NMR (100 MHz, DMSO-d₆) δ20.2, 21.1, 56.6, 57.0, 65.5, 66.2, 84.2, 84.9, 125.8, 126.1, 128.20, 128.22, 128.74, 128.76, 128.79, 128.9, 132.2, 135.3, 135.4, 138.2, 138.3, 138.3, 138.4, 156.1, 156.9, 175.9 and 177.1 ppm (diastereomeric signals).

### EXAMPLE 16

5-[Hydroxy-4-quinolin-3-yl-phenyl)-methyl-imidazolidine-2,4-dione

This compound was synthesised according to J. Org. Chem. 2001, 66, 1500-1502 from commercially available 3-bromo-quinoline and 5-[Hydroxy-(4-iodo-phenyl)-methyl]-imidazolidine-2,4-dione described above.

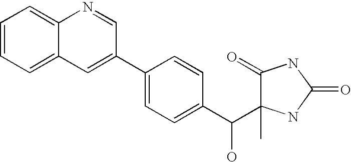

APCI-MS m/z: 348.2 [MH⁺]

What we claim is:

1. A compound of the formula I or a pharmaceutically acceptable salt or an in vivo hydrolysable ester thereof 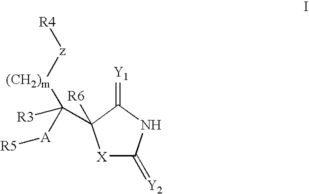
wherein
X is selected from NR1, O, S;
Y1 and Y2 are independently selected from O, S;
Z is selected from NR2, O, S;
m is 0 or 1;
A is selected from a direct bond, (C1-6)alkyl, (C1-6) alkenyl, (C1-6)haloalkyl, or (C1-6)heteroalkyl containing a hetero group selected from N, O, S, SO, SO2 or containing two hetero groups selected from N, O, S, SO, SO2 and separated by at least two carbon atoms;
R1 is selected from H, alkyl, haloalkyl;
R2 is selected from H, alkyl, haloalkyl;
R3 and R6 are independently selected from H, halogen (preferably F), alkyl, haloalkyl, alkoxyalkyl, heteroalkyl, cycloalkyl, aryl, alkyl-cycloalkyl, alkyl-heterocycloalkyl, heteroalkyl-cycloalkyl, heteroalkyl-heterocycloalkyl, cycloalkyl-alkyl, cycloalkyl-heteroalkyl, heterocycloalkyl-alkyl, heterocycloalkyl-heteroalkyl, alkylaryl, heteroalkyl-aryl, heteroaryl, alkylheteroaryl, heteroalkyl-heteroaryl, arylalkyl, aryl-heteroalkyl, heteroaryl-alkyl, heteroaryl-heteroalkyl, bisaryl, aryl-heteroaryl, heteroaryl-aryl, bisheteroaryl, cycloalkyl or heterocycloalkyl comprising 3 to 7 ring atoms, wherein the alkyl, heteroalkyl, aryl, heteroaryl, cycloalkyl or heterocycloalkyl radicals may be optionally substituted by one or more groups independently selected from hydroxy, alkyl, heteroalkyl, cycloalkyl, heterocycloalkyl, aryl, heteroaryl, halo, haloalkyl, hydroxyalkyl, alkoxy, alkoxyalkyl, haloalkoxy, haloalkoxyalkyl, carboxy, carboxyalkyl, alkylcarboxy, amino, N-alkylamino, N,N-dialkylamino, alkylamino, alkyl(N-alkyl)amino, alkyl(N,N-dialkyl)amino, amido, N-alkylamido, N,N-dialkylamido, alkylamido, alkyl(N-alkyl)amido, alkyl(N,N-dialkyl)amido, alkylcarbamate, alkylcarbamide, thiol, sulfone, sulfonamino, alkylsulfonamino, arylsulfonamino, sulfonamido, haloalkyl sulfone, alkylthio, arylthio, alkylsulfone, arylsulfone, aminosulfone, N-alkylaminosulfone, N,N-dialkylaminosulfone, alkylaminosulfone, arylaminosulfone, cyano, alkylcyano, guanidino, N-cyano-guanidino, thioguanidino, amidino, N-aminosulfon-amidino, nitro, alkylnitro, 2-nitro-ethene-1,1-diamine;
R4 is selected from H, alkyl, hydroxyalkyl, haloalkyl, alkoxyalkyl, haloalkoxy, aminoalkyl, amidoalkyl, thioalkyl;
R5 is a bicyclic or tricyclic group comprising two or three ring structures each of 3 to 7 ring atoms independently selected from cycloalkyl, aryl, heterocycloalkyl or heteroaryl, with each ring structure being independently optionally substituted by one or more substituents independently selected from halogen, thiolo, thioalkyl, hydroxy, alkylcarbonyl, haloalkoxy, amino, N-alkylamino, N,N-dialkylamino, cyano, nitro, alkyl, haloalkyl, alkoxy, alkyl sulfone, alkylsulfonamido, haloalkyl sulfone, alkylamido,alkylcarbamate, alkylcarbamide, carbonyl, carboxy, wherein any alkyl radical within any substituent may itself be optionally substituted by one or more groups independently selected from halogen, hydroxy, amino, N-alkylamino, N,N-dialkylamino, alkylsulfonamino, alkylcarboxyamino, cyano, nitro, thiol, alkylthiol, alkylsulfono, alkylaminosulfono, alkylcarboxylate, amido, N-alkylamido, N,N-dialkylamido, alkylcarbamate, alkylcarbamide, alkoxy, haloalkoxy, carbonyl, carboxy;
R5 is a bicyclic or tricyclic group wherein each ring structure is joined to the next ring structure by a direct bond, by —O—, by —S—, by —NH—, by (C1-6)alkyl, by (C1-6)haloalkyl, by (C1-6)heteroalkyl, by (C1-6)alkenyl, by (C1-6)alkynyl, by sulfone, by carboxy(C1-6)alkyl, or is fused to the next ring structure;
Optionally R2 and R4 may join to form a ring comprising up to 7 ring atoms or R3 and R6 may join to form a ring comprising up to 7 ring atoms;
Provided that:
when X is NR1, R1 is H, Y1 is O, Y2 is O, Z is O, m is 0, A is a direct bond, R3 is H, R4 is H and R6 is H, then R5 is not n-methylbenzimidazole, or 5-(benzo[1,3]dioxol-5-yl;
when X is S, at least one of Y1 and Y2 is O, m is 0, A is a direct bond, R3 is H or methyl, R6 is H or methyl, then R5 is not quinoxaline-1,4-dioxide.

2. A compound of the formula I as claimed in claim 1 or a pharmaceutically acceptable salt or an in vivo hydrolysable ester thereof, wherein X is NR1, R1 is H or (C1-3) alkyl, at least one of Y1 and Y2 is O, Z is O, m is 0, and A is a direct bond.

3. A compound as claimed in either claim 1 or claim 2 or a pharmaceutically acceptable salt or an in vivo hydrolysable ester thereof, wherein R3 is H, alkyl or haloalkyl, R4 is H, alkyl or haloalkyl.

4. A compound as claimed in any of the preceding claims or a pharmaceutically acceptable salt or an in vivo hydrolysable ester thereof, wherein R5 is a bicyclic group comprising two optionally substituted 5 or 6 membered rings independently selected from cycloalkyl, aryl, heterocycloalkyl or heteroaryl.

5. A compound as claimed in any of the preceding claims or a pharmaceutically acceptable salt or an in vivo hydrolysable ester thereof, wherein R6 is H, alkyl, hydroxyalkyl, aminoalkyl, cycloalkyl-alkyl, alkyl-cycloalkyl, arylalkyl, alkylaryl, heteroalkyl, heterocycloalkyl-alkyl, alkyl-heterocycloalkyl, heteroaryl-alkyl or heteroalkyl-aryl.

6. A compound of the formula Ib or a pharmaceutically acceptable salt or an in vivo hydrolysable ester thereof
Formula Ib: 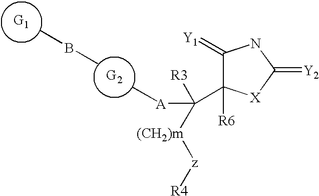
wherein
X is selected from NR1, O, S;
Y1 and Y2 are independently selected from O, S;
Z is selected from NR2, O, S;
m is 0 or 1;
A is selected from a direct bond, (C1-6)alkyl, (C1-6)haloalkyl, or (C1-6) heteroalkyl containing a hetero atom selected from O, S;
B is selected from a direct bond, —O—, —S—, —NH—, amide, carbamate, carbonyl, (C1-6)alkyl, (C1-6)haloalkyl, (C2-6)alkenyl, (C2-6)alkynyl, or (C1-6)heteroalkyl containing a hetero atom selected from O, S;
R1 is selected from H, (C1-3)alkyl or (C1-3)haloalkyl;
R2 is selected from H, (C1-3)alkyl or (C1-3)haloalkyl;
R3 is selected from H, (C1-3)alkyl or (C1-3)haloalkyl;
R4 is selected from H, (C1-3)alkyl or (C1-3)haloalkyl;
R6 is selected from H, alkyl, heteroalkyl, (C3-7)cycloalkyl, (C3-7)heterocycloalkyl, (C3-7)aryl, (C3-7)heteroaryl, alkyl-(C3-7)cycloalkyl, alkyl-(C3-7)heterocycloalkyl, alkyl-(C3-7)aryl, alkyl-(C3-7)heteroaryl, heteroalkyl-(C3-7)cycloalkyl, heteroalkyl-(C3-7)heterocycloalkyl, heteroalkyl-(C3-7)aryl, heteroalkyl-(C3-7)heteroaryl, (C3-7)cycloalkyl-alkyl, (C3-7)heterocycloalkyl-alkyl, (C3-7)ary-alkyl, (C3-7)heteroaryl-alkyl, (C3-7)cycloalkyl-heteroalkyl, (C3-7)heterocycloalkyl-heteroalkyl, (C3-7)aryl-heteroalkyl, (C3-7)heteroaryl-heteroalkyl;
in R6 the alkyl, heteroalkyl, aryl, heteroaryl, cycloalkyl or heterocycloalkyl radicals may be optionally substituted by one or more groups independently selected from hydroxy, alkyl,halo, haloalkyl, hydroxyalkyl, alkoxy, alkoxyalkyl, haloalkoxy, haloalkoxyalkyl, carboxy, carboxyalkyl, alkylcarboxy, amino, N-alkylamino, N,N-dialkylamino, alkylamino,alkyl(N-alkyl)amino, alkyl(N,N-dialkyl)amido, amido, N-alkylamido, N,N-dialkylamido, alkylamido, alkyl(N-alkyl)amido, alkyl(N,N-dialkyl)amido, alkylcarbamate, alkylcarbamide, thiol, sulfone, sulfonamino, alkylsulfonamino, arylsulfonamino, sulfonamido, haloalkyl sulfone, alkylthio, arylthio, alkylsulfone, arylsulfone, aminosulfone, N-alkylaminosulfon, N,N-dialkylaminosulfone, alkylaminosulfone, arylaminosulfone, cyano, alkylcyano, guanidino, N-cyano-guanidino, thioguanidino, amidino, N-aminosulfon-amidino, nitro, alkylnitro, 2-nitro-ethene-1,1-diamine;
either G1 is a monocyclic group and G2 is selected from a monocyclic group and a bicyclic group, or G1 is a bicyclic group and G2 is a monocyclic group, wherein the monocyclic group comprises one ring structure and the bicyclic group comprises two ring structures either fused together or joined together by B as defined above, each ring structure having up to 7 ring atoms and being independently selected from cycloalkyl, aryl, heterocycloalkyl or heteroaryl, with each ring structure being independently optionally substituted by one or more substituents independently selected from halogen, thiolo, thioalkyl, hydroxy, alkylcarbonyl, haloalkoxy, amino, N-alkylamino, N,N-dialkylamino, cyano, nitro, alkyl, haloalkyl alkoxy, alkyl sulfone,alkylsulfonamido, haloalkyl sulfone, alkylamido,alkylcarbamate, alkylcarbamide, wherein any alkyl radical-within any substituent may itself be optionally substituted by one or more groups independently selected from halogen, hydroxy, amino, N-alkylamino, N,N-dialkylamino, alkylsulfonamino, cyano, nitro, thiol, alkylthiol, alkylsulfono, alkylaminosulfono, alkylcarboxylate, amido, N-alkylamido, N,N-dialkylamido, alkylcarbamate, alkylcarbamide,alkoxy, haloalkoxy;
Optionally R3 and R6 may join to form a ring comprising up to 7 ring atoms.

7. A compound of the formula Ib as claimed in claim 6 or a pharmaceutically acceptable salt or an in vivo hydrolysable ester thereof, wherein X is NR1; at least one of Y1 and Y2 is O; Z is O; m is 0; A is a direct bond, (C1-6)alkyl or (C1-6)heteroalkyl containing a hetero atom selected from O, S; B is a direct bond, acetylene, CON (amide), (C1-C4)alkyloxy, —O—, —S— or —NH—; R1 is H or methyl; R3 is H, (C1-3)alkyl or (C1-3)haloalkyl; R4 is H, (C1-3)alkyl or (C1-3)haloalkyl.

8. A compound of the formula Ib as claimed in claim 6 or a pharmaceutically acceptable salt or an in vivo hydrolysable ester thereof, wherein X is NR1 and R1 is H; and Y1 and Y2 is are each O; and Z is O; and m is 0; and A is a direct bond; and B is selected from a direct bond, acetylene, —O—, —NH—, —S—, or CH₂O; and R3 is H; and R4 is H.

9. A compound of the formula Ic or a pharmaceutically acceptable salt or an in vivo hydrolysable ester thereof
Formula Ic: 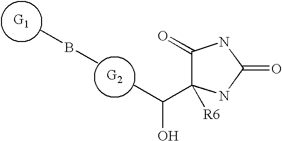
wherein
B is selected from a direct bond, acetylene, —O—, —NH—, —S—, or CH₂O;
R6 is selected from H, alkyl, heteroalkyl, (C3-7)cycloalkyl, (C3-7)heterocycloalkyl, (C3-7)aryl, (C3-7)heteroaryl, alkyl-(C3-7)cycloalkyl, alkyl-(C3-7)heterocycloalkyl, alkyl-(C3-7)aryl, alkyl-(C3-7)heteroaryl, heteroalkyl-(C3-7)cycloalkyl, heteroalkyl-(C3-7)heterocycloalkyl, heteroalkyl-(C3-7)aryl, heteroalkyl-(C3-7)heteroaryl, (C3-7)cycloalkyl-alkyl, (C3-7)heterocycloalkyl-alkyl, (C3-7)ary-alkyl, (C3-7)heteroaryl-alkyl, (C3-7)cycloalkyl-heteroalkyl, (C3-7)heterocycloalkyl-heteroalkyl, (C3-7)aryl-heteroalkyl, (C3-7)heteroaryl-heteroalkyl;
in R6 the alkyl, heteroalkyl, aryl, heteroaryl, cycloalkyl or heterocycloalkyl radicals may be optionally substituted by one or more groups independently selected from hydroxy, alkyl, halo, haloalkyl, hydroxyalkyl, alkoxy, alkoxyalkyl, haloalkoxy, haloalkoxyalkyl, carboxy, carboxyalkyl, alkylcarboxy, amino, N-alkylamino, N,N-dialkylamino, alkylamino,alkyl(N-alkyl)amino, alkyl(N,N-dialkyl)amino, amido, N-alkylamido, N,N-dialkylamido, alkylamido, alkyl(N-alkyl)amido, alkyl(N,N-dialkyl)amido, alkylcarbamate, alkylcarbamide, thiol, sulfone, sulfonamino, alkylsulfonamino, arylsulfonamino, sulfonamido, haloalkyl sulfone, alkylthio, arylthio, alkylsulfone, arylsulfone, aminosulfone, N-alkylaminosulfon, N,N-dialkylaminosulfone, alkylaminosulfone, arylaminosulfone, cyano, alkylcyano, guanidino, N-cyano-guanidino, thioguanidino, amidino, N-aminosulfon-amidino, nitro, alkylnitro, 2-nitro-ethene-1,1-diamine;
either G1 is a monocyclic group and G2 is selected from a monocyclic group and a bicyclic group, or G1 is a bicyclic group and G2 is a monocyclic group, wherein the monocyclic group comprises one ring structure and the bicyclic group comprises two ring structures either fused together or joined together by B as defined above, each ring structure having up to 7 ring atoms and being independently selected from cycloalkyl, aryl, heterocycloalkyl or heteroaryl, with each ring structure being independently optionally substituted by one or more substituents independently selected from halogen, thiolo, thioalkyl, hydroxy, alkylcarbonyl, haloalkoxy, amino, N-alkylamino, N,N-dialkylamino, cyano, nitro, alkyl, haloalkyl alkoxy, alkyl sulfone,alkylsulfonamido, haloalkyl sulfone, alkylamido,alkylcarbamate, alkylcarbamide, wherein any alkyl radical within any substituent may itself be optionally substituted by one or more groups independently selected from halogen, hydroxy, amino, N-alkylamino, N,N-dialkylamino, alkylsulfonamino, cyano, nitro, thiol, alkylthiol, alkylsulfono, alkylaminosulfono, alkylcarboxylate, amido, N-alkylamido, N,N-dialkylamido, alkylcarbamate, alkylcarbamide,alkoxy, haloalkoxy.

10. A compound of the formula Ic as claimed in claim 9 or a pharmaceutically acceptable salt or an in vivo hydrolysable ester thereof, wherein B is selected from a direct bond, —O—, —S—, or CH₂O; G2 is a monocyclic group comprising an aryl ring; G1 is a monocyclic or bicyclic group comprising at least one aryl ring; R6 is selected from H, (C1-6)alkyl, (C1-6)heteroalkyl, heterocycloalkyl, heterocycloalkyl-(C1-6)alkyl, heteroaryl or heteroaryl-(C1-6)alkyl; in R6 the alkyl, heteroalkyl, aryl, heteroaryl, cycloalkyl or heterocycloalkyl radicals may be optionally substituted by one or more groups.

11. A compound of the formula Id or a pharmaceutically acceptable salt or an in vivo hydrolysable ester thereof
Formula Id: 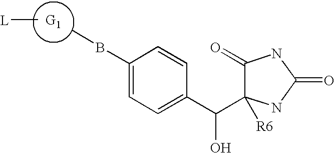
wherein
B is selected from a direct bond, O or CH₂O;
G1 is a monocyclic or bicyclic group comprising at least one five or six membered aryl ring;
R6 is H, alkyl, hydroxyalkyl, aminoalkyl, alkyl-carbamic acid alkyl ester, alkyl-alkyl-urea, alkylsulfonyl-alkyl, N-alkyl-alkylsulfonamide, heteroaryl-alkyl;
L is selected from H, alkyl, haloalkyl, hydroxy, alkoxy, haloalkoxy, amino, alkylamino, amido, alkylamido, alkylcarbamate, alkylcarbamide, alkylsulfono, alkylsulfonamido,nitro, cyano, halo;
or L is a group:
T—U—V—
wherein V is attached to G1 and V is selected from CH₂, O, NCO, NCOO, NCON or NSO₂;
U is (C1-5)alkyl;
T is selected from hydroxy, alkoxy, cyano, amino, alkylamino, alkylsulfono, alkylsulfonamide, alkylcarbamate, alkylacarbamide, alkylamide, imidazolyl, triazolyl or pyrollidon.

12. A compound of the formula Id as claimed in claim 11 or a pharmaceutically acceptable salt or an in vivo hydrolysable ester thereof, wherein G1 is selected from is phenyl, pyridyl, napthyl or quinoline.

13. A compound of the formula Id as claimed in either claim 11 or claim 12 or a pharmaceutically acceptable salt or an in vivo hydrolysable ester thereof, wherein R6 is selected from H, (C1-6)alkyl, hydroxy-(C1-6)alkyl, amino-(C1-6)alkyl, or heteoraryl-(C1-6)alkyl.

14. A compound ofthe formula Id as claimed in any of claims 11 to 13 or a pharmaceutically acceptable salt or an in vivo hydrolysable ester thereof, wherein L is selected from H, (C1-5)alkyl, (C1-5)haloalkyl, hydroxy, alkoxy, haloalkoxy, amino, (C1-5)alkylamino, amido, (C1-5)alkylamido, (C1-5)alkylcarbamate, (C1-5)alkylcarbamide, (C1-5)alkylsulfono, (C1-5)alkylsulfonamido, nitro, cyano, halo; or L is the group T—U—V— wherein U is unbranced (C1-5)alkyl, and T is selected from hydroxy, alkoxy, cyano, amino, (C1-3)alkylamino, (C1-3)alkylsulfono, (C1-3)alkylsulfonamide, (C1-3)alkylcarbamate, (C1-3)alkylacarbamide, (C1-3)alkylamide, imidazolyl, triazolyl or pyrollidon.

15. A compound of the formula Id as claimed in any of claims 11 to 14 or a pharmaceutically acceptable salt or an in vivo hydrolysable ester thereof, wherein L is a meta or para substituent and G1 is a 6 membered ring.

16. A pharmaceutical composition which comprises a compound of the formula I as claimed in claim 1 or a compound of the formula Ib as claimed in claim 6 or a compound of the formula Ic as claimed in claim 9 or a compound of the formula Id as claimed in claim 11 or a pharmaceutically acceptable salt or an in vivo hydrolysable ester thereof and a pharmaceutically acceptable carrier.

17. A method of treating a metalloproteinase mediated disease or condition which comprises administering to a warm-blooded animal a therapeutically effective amount of a compound of the formulae I or Ib or Ic or Id or a pharmaceutically acceptable salt or in vivo hydrolysable ester thereof.

18. Use of a compound of the formulae I or Ib or Ic or Id or a pharmaceutically acceptable salt or in vivo hydrolysable precursor thereof in the preparation of a medicament for use in the treatment of a disease or condition mediated by one or more metalloproteinase enzymes.
